(19) United States
(12) Patent Application Publication
(10) Pub. No.: US 2019/0048556 A1
(43) Pub. Date: Feb. 14, 2019

(54) SOIL QUALITY DETERMINATION DEVICE, SOIL QUALITY DETERMINATION METHOD, AND RECORDING MEDIUM HAVING PROGRAM STORED THEREON

(71) Applicant: NEC Corporation, Minato-ku, Tokyo (JP)

(72) Inventor: Shinji Kasahara, Tokyo (JP)

(73) Assignee: NEC Corporation, Minato-ku, Tokyo (JP)

(21) Appl. No.: 15/763,534

(22) PCT Filed: Sep. 14, 2016
(86) PCT No.: PCT/JP2016/004192

§ 371 Date: Mar. 27, 2018

(30) Foreign Application Priority Data
Sep. 30, 2015 (JP) 2015-193107

(51) Int. Cl. E02D 33/00; G01N 33/24

(52) U.S. Cl. CPC E02D 33/00; G01N 33/246

(57) ABSTRACT

Provided is a technology for soil quality determination that makes it possible to calculate the safety rate of a slope without determining the soil properties of soil to be measured beforehand. A soil quality determination device 110B according to an embodiment of the present invention is provided with a vibration feature value calculation unit 103 for calculating a vibration feature value on the basis of vibration data expressing the vibration of given soil subjected to vibration while having water repeatedly added thereto and a soil quality determination unit 105 for determining the quality of the given soil on the basis of a water feature value distribution for the given soil expressing the relationship between the amount of water measured during the acquisition of the vibration data and the vibration feature value, the degree of similarity between the water feature value distributions of a soil type that is a type of soil from which the water feature value distribution is obtained and the given soil, and the properties of the soil type.

### TECHNICAL FIELD

The present invention relates to a technology of determining a soil quality of a monitoring target.

### BACKGROUND ART

PTL 1 describes an example of a technology of determining a quality of a soil. In PTL 1, a curve indicating a dry density-volume water content relation of a soil used at a construction site is previously prepared. A volume water content is measured at the construction site, based on a characteristic of a transmitted electromagnetic wave obtained by transmitting an electromagnetic wave through earth. A determination device in PTL 1 estimates a dry density of the earth at the construction site, based on the previously prepared dry density-volume water content curve.

### CITATION LIST

### Patent Literature

[PTL 1] Japanese Unexamined Patent Application Publication No. 2007-010568

### SUMMARY OF INVENTION

### Technical Problem

For example, the dry density-volume water content relation used by the determination device in PTL 1 is obtained by an experiment using a soil used at the construction site. The dry density-volume water content relation obtained by the experiment using the soil used at the construction site holds only for the soil used at the construction site. The dry density-volume water content relation in the soil used at the construction site does not hold for another type of soil. In the technology in PTL 1, in order to estimate dry densities for a plurality of types of soil, a dry density-volume water content relation needs to be obtained for each of the soil types.

An object of the present invention is to provide a soil quality determination technology capable of calculating a safety factor of a slope without previously obtaining a quality of a measurement target soil.

### Solution to Problem

A soil quality determination device according to an aspect of the present invention includes: vibration feature value calculation means for calculating a vibration feature value, based on vibration data representing vibration of a target soil to which vibration is applied with repeated water addition; and soil quality determination means for determining a quality of the target soil, based on a water amount-feature value distribution of the target soil, the distribution representing a relation between a water amount measured when the vibration data are acquired and the vibration feature value, an extent of similarity of the water amount-feature value distribution between a soil type being a type of a soil from which the water amount-feature value distribution is obtained and the target soil, and a quality of the soil type.

A soil quality determination method according to an aspect of the present invention includes: calculating a vibration feature value, based on vibration data representing vibration of a target soil to which vibration is applied with repeated water addition; and determining a quality of the target soil, based on a water amount-feature value distribution of the target soil, the distribution representing a relation between a water amount measured when the vibration data are acquired and the vibration feature value, an extent of similarity of the water amount-feature value distribution between a soil type being a type of a soil from which the water amount-feature value distribution is obtained and the target soil, and a quality of the soil type.

A recording medium according to an aspect of the present invention stores a soil quality determination program causing a computer to execute: vibration feature value calculation processing of calculating a vibration feature value, based on vibration data representing vibration of a target soil to which vibration is applied with repeated water addition; and soil quality determination processing of determining a quality of the target soil, based on a water amount-feature value distribution of the target soil, the distribution representing a relation between a water amount measured when the vibration data are acquired and the vibration feature value, an extent of similarity of the water amount-feature value distribution between a soil type being a type of a soil from which the water amount-feature value distribution is obtained and the target soil, and a quality of the soil type. An aspect of the present invention can be achieved by the soil quality determination program described above.

### Advantageous Effects of Invention

The present invention provides an effect that a safety factor of a slope can be calculated without previously obtaining a quality of a measurement target soil.

### BRIEF DESCRIPTION OF DRAWINGS

FIG. 1 is a block diagram illustrating a configuration of a soil quality determination system according to a first example embodiment of the present invention.

FIG. 2 is a flowchart illustrating an operation example of the soil quality determination system according to the first example embodiment of the present invention.

FIG. 3 is a block diagram illustrating a configuration of a soil quality determination system according to a second example embodiment of the present invention.

FIG. 4 is a flowchart illustrating an operation example of the soil quality determination system according to the second example embodiment of the present invention.

FIG. 5 is a block diagram illustrating a configuration of a detection system according to a third example embodiment of the present invention.

FIG. 6 is a flowchart illustrating an operation example of the detection system according to the third example embodiment of the present invention.

FIG. 7 is a flowchart illustrating another operation example of the detection system according to the third example embodiment of the present invention.

FIG. 8 is a flowchart illustrating an operation example of a triaxial compression test by the detection system according to the third example embodiment of the present invention.

FIG. 9 is a flowchart illustrating an operation example of processing in a water addition excitation test by the detection system according to the third example embodiment of the present invention.

FIG. 10 is a diagram illustrating a configuration example of a soil quality determination system according to a fourth example embodiment of the present invention.

FIG. 11 is a diagram illustrating an overall operation of the soil quality determination device according to the fourth example embodiment of the present invention.

FIG. 12 is a flowchart illustrating an operation example of comparison processing of a damping factor-water amount distribution by the soil quality determination device according to the fourth example embodiment of the present invention.

FIG. 13 is a diagram schematically illustrating an example of a stored degree of similarity.

FIG. 14 is a block diagram illustrating a configuration example of a soil quality determination device 110B according to a fifth example embodiment of the present invention.

FIG. 15 is a diagram illustrating an example of a hardware configuration of a computer capable of providing the soil quality determination device and the detection device, according to the respective example embodiments of the present invention.

FIG. 16 is a block diagram illustrating a configuration example of the soil quality determination device according to the first, second and fourth example embodiments of the present invention, the device being implemented by use of dedicated circuits.

FIG. 17 is a block diagram illustrating a configuration example of the detection device according to the third example embodiment of the present invention, the device being implemented by use of dedicated circuits.

FIG. 18 is a block diagram illustrating a configuration example of the soil quality determination device according to the fifth example embodiment of the present invention, the device being implemented by use of dedicated circuits.

### DESCRIPTION OF EMBODIMENTS

Example embodiments of the present invention will be described in detail below with reference to drawings. First, a principle of slope failure precursor detection used in each example embodiment of the present invention will be described, and then the example embodiments will be described.

### Principle of Slope Failure Precursor Detection

Stability of a slope may be evaluated using a relation between a shearing stress acting in a sloping direction and a shearing strength preventing a slide caused by the shearing stress. The shearing stress may be expressed by gravity acting on earth and sand, and a slope gradient angle. The shearing strength may be classified into an adhesive strength possessed by earth and a resistance force based on a normal stress. The earth may be hereinafter also simply expressed as a “soil.” A lump of soil is expressed as a “clod.” The aforementioned normal stress is determined by gravity acting on a clod and a slope gradient angle. The resistance force is determined by the normal stress and an effective friction coefficient. The clod contains particles of soil (also hereinafter expressed as “soil particles”), and pore air and pore water that exist in a gap between particles. A normal reaction by soil particles, a pore air pressure, and a pore water pressure act as a reaction supporting a weight of the clod. However, out of the forces, only the normal reaction by soil particles contributes to the shearing strength. Accordingly, when the shearing strength is calculated, an apparent normal stress obtained by subtracting the pore water pressure and the pore air pressure from gravity shall be used. As a water content increases, the apparent normal stress decreases. Additionally, it is also known that values of the effective friction coefficient and the adhesive strength decrease with increase in the water content of the earth. The effective friction coefficient evaluated by being multiplied by the normal stress, and the adhesive strength are coefficients set in such a way that the shearing stress and the shearing strength balance when a slope slides. The aforementioned resistance force is determined by a product of the effective friction coefficient and the aforementioned apparent normal stress. Accordingly, as the water content of the earth increases, the shearing stress increases, and the shearing strength decreases, thus causing a slope failure.

It can be understood from the description above that a slope failure can be predicted based on increase in a water content. In a method employed in the example embodiments of the present invention described below, a vibration damping factor or a soil water amount is detected in place of the water content. Further, a parameter affecting a shearing strength and a shearing stress that change with the water content is previously measured with respect to earth with a plurality of different soil qualities. The result of the previously performed measurement is stored in a database as a distribution in an earth model, the distribution being related to a vibration damping factor or a soil water amount. Then, based on the previously performed measurement result and a measurement result on a measurement target, a soil quality determination system according to the example embodiments of the present invention estimates a soil quality of a soil in the measurement target being and selects a model to be used for safety monitoring.

### First Example Embodiment

### Configuration of First Example Embodiment

Next, a soil quality determination system 100 according to a first example embodiment of the present invention will be described in detail with reference to drawings.

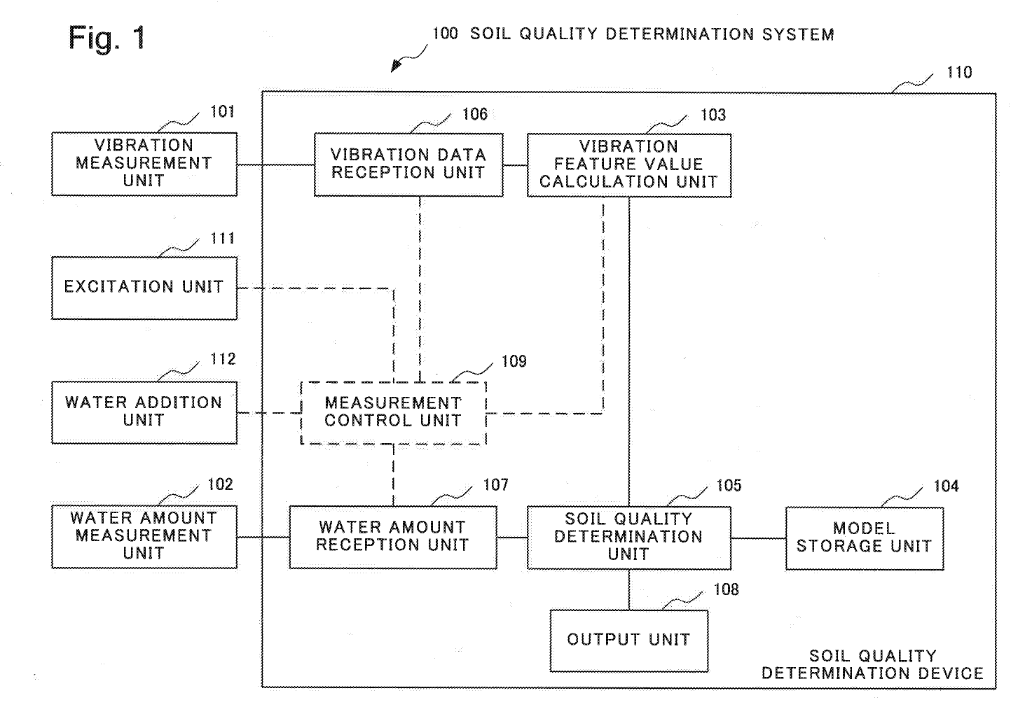

FIG. 1 is a block diagram illustrating a configuration of the soil quality determination system 100 according to the first example embodiment of the present invention. As illustrated in FIG. 1, the soil quality determination system 100 according to the present example embodiment includes a vibration measurement unit 101, a water amount measurement unit 102, a vibration feature value calculation unit 103, a model storage unit 104, a soil quality determination unit 105, a vibration data reception unit 106, a water amount reception unit 107, and an output unit 108. In the example illustrated in FIG. 1, the soil quality determination system 100 includes a soil quality determination device 110. Then, the soil quality determination device 110 includes the vibration feature value calculation unit 103, the model storage unit 104, the soil quality determination unit 105, the vibration data reception unit 106, the water amount reception unit 107, and the output unit 108. Further, the soil quality determination device 110 is connected to the vibration measurement unit 101 and the water amount measurement unit 102.

The soil quality determination system 100 may further include an excitation unit 111 and a water addition unit 112. In that case, the soil quality determination device 110 may further include a measurement control unit 109. Then, the soil quality determination device 110 may be further connected to the excitation unit 111 and the water addition unit 112.

The vibration measurement unit 101 detects (i.e. performs sensing on) vibration of a measurement target soil. The vibration measurement unit 101 outputs vibration data representing the detected vibration as, for example, a signal to the vibration data reception unit 106. For example, the vibration data are time-series data representing vibration. Specifically, the vibration data are data such as a position, a speed, an acceleration, or a pressure of the measurement target soil, the data being measured at every predetermined time. The vibration data may be another type of time-series data. For example, the vibration measurement unit 101 is a vibration sensor detecting (i.e. performing sensing on) vibration of a measurement target soil and outputting vibration data representing the detected vibration as a signal. Various existing sensors detecting vibration are applicable as the vibration sensor. In the respective example embodiments of the present invention, a type of soil is expressed as a “soil type.” A measurement target soil is expressed as a “target soil” or an “estimated target soil.” Further, a type of target soil is expressed as a “target soil type” or an “estimated target soil type.”

The vibration data reception unit 106 receives vibration data representing vibration from the vibration measurement unit 101. The vibration data reception unit 106 transmits the received vibration data to the vibration feature value calculation unit 103. The vibration data reception unit 106 may convert a signal representing the vibration data, the signal being output by the vibration measurement unit 101, into vibration data recognizable to the vibration feature value calculation unit 103. Then, the vibration data reception unit 106 may transmit the vibration data obtained by the conversion to the vibration feature value calculation unit 103.

The water amount measurement unit 102 measures a water amount of a target soil. The water amount measurement unit 102 outputs data representing the measured water amount as a signal to, for example, the water amount reception unit 107. For example, the water amount is a ratio of a weight of water contained in a soil. The water amount may be another value. For example, the water amount measurement unit 102 is a sensor measuring a water amount of a target soil and outputting data representing the measured water amount as a signal. Such a sensor is also expressed as a moisture meter. Various existing sensors measuring a water amount in a soil are applicable as the water amount measurement unit 102.

The water amount reception unit 107 receives a water amount from the water amount measurement unit 102. The water amount reception unit 107 transmits the received water amount to the soil quality determination unit 105. The water amount reception unit 107 may convert a signal representing the water amount, the signal being output by the water amount measurement unit 102, into data representing the water amount in a form recognizable to the soil quality determination unit 105. Then, the water amount reception unit 107 may transmit the water amount data obtained by the conversion to the soil quality determination unit 105.

The vibration feature value calculation unit 103 receives time-series data on vibration detected by the vibration measurement unit 101 through, for example, the vibration data reception unit 106. Based on the received time-series data on the vibration, the vibration feature value calculation unit 103 calculates a feature value representing a feature of the vibration of the target soil.

For example, the vibration feature value is a damping factor. Various existing methods are applicable as the calculation method of a damping factor from the time-series data on the vibration by the vibration feature value calculation unit 103. For example, the vibration feature value calculation unit 103 may calculate a damping factor, based on a difference between peaks in the time-series data on the vibration. Further, the vibration feature value calculation unit 103 may convert the time-series data on the vibration into a frequency domain. Then, the vibration feature value calculation unit 103 may calculate a peak frequency, peak power, and a half-value width with respect to the peak power and may calculate a damping factor, based on the calculated values.

For example, the excitation unit 111 is a device capable of applying vibration to a target soil through an operation by an operator.

For example, the water addition unit 112 is a device capable of adding a predetermined amount of water to a target soil through an operation by an operator.

An operator performs vibration measurement measuring vibration of a target soil by the vibration measurement unit 101, while applying vibration to the target soil by the excitation unit 111. Additionally, the operator performs moisture measurement measuring a water amount of the target soil by the water amount measurement unit 102. Next, by use of the water addition unit 112, the operator increases water contained in the target soil by, for example, performing water addition through adding a predetermined amount of water to the target soil. The operator performs vibration measurement and moisture measurement on the target soil with an increased amount of contained water. The operator repeats water addition, and vibration measurement and moisture measurement until the water amount contained in the target soil exceeds a threshold value.

As described above, the soil quality determination device 110 may include the measurement control unit 109. In that case, the measurement control unit 109 may provide the excitation unit 111 with an instruction to apply vibration to the target soil and an instruction to stop applying vibration. In that case, the excitation unit 111 may be implemented to apply vibration to the target soil in accordance with an instruction from the measurement control unit 109. The excitation unit 111 may be implemented to apply vibration in a predetermined vibration pattern for a certain period of time. The excitation unit 111 may be implemented to stop applying vibration in accordance with an instruction from the measurement control unit 109. The measurement control unit 109 may notify the vibration data reception unit 106 or the vibration feature value calculation unit 103 of transmission of an instruction to apply vibration to the target soil. The measurement control unit 109 may notify the vibration data reception unit 106 or the vibration feature value calculation unit 103 of transmission of an instruction to stop applying vibration to the target soil. The vibration data reception unit 106 may receive vibration data while vibration is being applied in accordance with an instruction by the measurement control unit 109. The vibration data reception unit 106 may calculate a vibration feature value, based on the vibration data received while vibration is being applied in accordance with the instruction by the measurement control unit 109.

The measurement control unit 109 may instruct the water addition unit 112 to add water to the target soil. In that case, for example, the water addition unit 112 may be implemented to add a certain amount of water to the target soil in accordance with an instruction from the measurement control unit 109. The measurement control unit 109 may notify the water amount reception unit 107 of transmission of an instruction for water addition to the target soil. The water amount reception unit 107 may receive a water amount from the water amount measurement unit 102 after the instruction for water addition to the target soil is provided, such as after a predetermined time elapses.

The vibration measurement unit 101 may measure vibration of a clod a plurality of number of times in a state where the water amount contained in the clod is the same. The vibration data reception unit 106 may receive a plurality of sets of vibration data in a state where the water amount is the same. The vibration feature value calculation unit 103 may calculate a vibration feature value from each of the plurality of sets of vibration data measured in a state where the water amount is the same. The vibration feature value calculation unit 103 may perform statistical processing of calculating a representative value such as calculation of an average value, calculation of a median value, or calculation of another statistical value on the calculated vibration feature values.

The water amount measurement unit 102 may measure a water amount a plurality of number of times in a state where the water amount contained in a clod is the same. The water amount reception unit 107 may receive a plurality of water amounts measured in a state where the water amount contained in the clod is the same. The soil quality determination unit 105 may perform, for example, the aforementioned statistical processing on the plurality of water amounts measured in a state where the water amount contained in the clod is the same.

In the following description, for example, a combination of a type of soil (i.e. a soil type) and a condition at the time of measurement is expressed as a “model” or a “model soil type.” For example, the condition at the time of measurement may be a density of the soil. Data representing a feature of a model soil type is expressed as “model data.” A quality of a soil is expressed as a “soil quality.” A “soil quality model” refers to data specifying a soil quality. The soil quality model is expressed by function expression modeling parameters (e.g. an adhesive strength, an internal friction angle, a clod weight, and a pore water pressure) required for a slope stability analysis formula, based on, for example, a vibration feature value, or a parameter such as a coefficient specifying the function expression. For example, modeling based on a vibration feature value and the like refers to specifying a relational expression when a parameter is expressed as the relational expression with the vibration feature value and the like as variables. The model data include a soil quality model, and a distribution of a combination of a water amount and a vibration feature value. The distribution of a combination of a water amount and a vibration feature value is a distribution of a combination of a vibration feature value calculated based on a measurement result of vibration of a soil, and a measurement result of a water amount of the soil in which the vibration is measured.

In descriptions of the respective example embodiments of the present invention, a model is a combination of a type of a soil and a density of the soil. A vibration feature value is a damping factor. A distribution of a combination of a water amount and a vibration feature value is a damping factor-water amount distribution.

For each combination of a soil type and a density (i.e. for each model), the model storage unit 104 stores data (i.e. model data) representing a feature of a soil of the soil type at the density, in a form of, for example, a database. For example, as the model data, the model storage unit 104 stores a function expression that, based on a vibration feature value, models a parameter required for a slope stability analysis formula, and a distribution of a vibration feature value with respect to a soil water amount. A form of the aforementioned function expression may be predetermined. Additionally, as the soil quality model, the model storage unit 104 may store a parameter, such as a coefficient, specifying the function expression instead of the function expression itself. As described above, the vibration feature value according to the present example embodiment is, for example, a damping factor. When the vibration feature value is a damping factor, the distribution of a vibration feature value with respect to a soil water amount is also expressed as a “damping factor-water amount distribution.”

Based on a damping factor calculated by use of vibration data measured with a plurality of different water amounts added to a target soil and a measured water amount of the target soil, the soil quality determination unit 105 derives a relation between the damping factor and the water amount (a damping factor-water amount distribution) of the target soil. The soil quality determination unit 105 selects at least one model, based on similarity between the damping factor-water amount distribution of the target soil and a damping factor-water amount distribution of a model, the distribution of the model being stored in the model storage unit 104.

Specifically, for example, the soil quality determination unit 105 calculates a degree of similarity (the “degree of similarity” may be hereinafter also expressed as a “score”) indicating an extent of similarity between the damping factor-water amount distribution of the target soil and a damping factor-water amount distribution of each model, the distribution of each model being stored in the model storage unit 104. The soil quality determination unit 105 may calculate a distance between a damping factor-water amount distribution of a model and the damping factor-water amount distribution of the target soil as a degree of similarity of the damping factor-water amount distribution of the model. For example, the distance may be a root sum square of differences between damping factors in a state where the water amount is the same. The distance may be a distance based on another definition. For example, the degree of similarity may be a value, such as a reciprocal of a distance, indicating that a greater value of a degree of similarity between the target soil and a model represents higher similarity between the target soil and the model, that is, better similarity between the target soil and the model. In that case, a sufficiently large value may be defined as a degree of similarity when a distance is zero. The soil quality determination unit 105 may calculate a regression equation representing a damping factor-water amount distribution, based on the damping factor-water amount distribution. The soil quality determination unit 105 may calculate a degree of similarity, based on a parameter of a regression equation of the target soil and a parameter of a function expression of a soil quality model of a model soil type. The soil quality determination unit 105 may calculate a degree of similarity by another method calculating an extent of similarity between distributions.

Then, based on the calculated degree of similarity, the soil quality determination unit 105 may select a model soil type having the closest damping factor-water amount distribution to the damping factor-water amount distribution of the target soil.

Based on a parameter of a soil quality model of the selected model soil type, the soil quality determination unit 105 determines a parameter of a monitoring model of the target soil. For example, when a soil type is selected, the soil quality determination unit 105 determines a parameter of a soil quality model of the selected model soil type to be a parameter of a monitoring model of the target soil. The parameter of the soil quality model is a parameter of the aforementioned function expression.

Based on the calculated degree of similarity, the soil quality determination unit 105 may select one or more damping factor-water amount distributions closest to the damping factor-water amount distribution of the target soil and may select one or more models having the selected damping factor-water amount distribution. For example, the method of selecting one or more models is as follows. In the following description, a greater value of a degree of similarity between the target soil and a model represents higher similarity between the target soil and the model, that is, better similarity between the target soil and the model. For example, the soil quality determination unit 105 may select predetermined number of models with damping factor-water amount distributions in descending order of the calculated degree of similarity. For example, the soil quality determination unit 105 may select models with damping factor-water amount distributions each having the calculated degree of similarity greater than or equal to a predetermined value. For example, the soil quality determination unit 105 may select models with damping factor-water amount distributions each having the calculated degree of similarity greater than or equal to a predetermined value, out of a predetermined number of models with damping factor-water amount distributions selected in descending order of the calculated degree of similarity. The soil quality determination unit 105 may select one or more models by a method other than the methods described above.

When a plurality of models are selected, the soil quality determination unit 105 may determine a weight based on a score for each model. The soil quality determination unit 105 may make a determination in such a way that a weight becomes greater as a score of a model becomes greater (i.e. as a damping factor-water amount distribution of a model becomes closer to the damping factor-water amount distribution of the target soil). The soil quality determination unit 105 may determine a sum of parameters multiplied by the determined weights of the selected models to be a parameter of a monitoring model of the target soil. The soil quality determination unit 105 may determine a density of the target soil in addition to the parameter of the aforementioned monitoring model. For example, the soil quality determination unit 105 may determine the density of the target soil by multiplying a density of a selected model by the weight determined for the model and adding up the densities multiplied by the weights.

The output unit 108 outputs a monitoring parameter of a target soil, the parameter being determined by the soil quality determination unit 105, to, for example, a display device (unillustrated) or a monitoring device (unillustrated).

### Operation of First Example Embodiment

Next, an operation of the soil quality determination system 100 according to the present example embodiment will be described in detail with reference to a drawing.

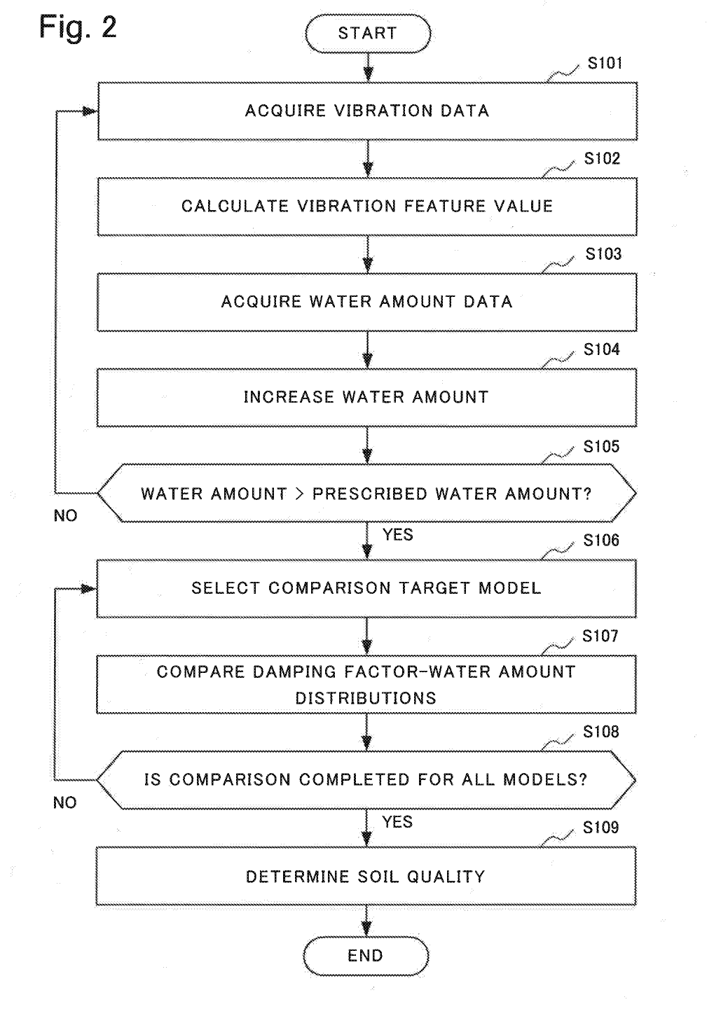

FIG. 2 is a flowchart illustrating an operation example of the soil quality determination system 100 according to the present example embodiment.

When the operation illustrated in FIG. 2 is started, the excitation unit 111 applies vibration to a target soil. The excitation unit 111 may apply vibration based on a vibration pattern predetermined to include vibrations at various frequencies to the target soil. Then, the vibration measurement unit 101 detects (i.e. performs sensing on) vibration of the target soil to which the vibration is applied. The vibration data reception unit 106 acquires time-series data representing the vibration detected by the vibration measurement unit 101 from the vibration measurement unit 101 (Step S101).

Next, the vibration feature value calculation unit 103 calculates a vibration feature value from the time-series data (i.e. vibration data) representing the vibration of the target soil, the data being received from the vibration measurement unit 101 (Step S102). In Step S101, the vibration measurement unit 101 may perform a plurality of number of measurements of vibration of the target soil containing a same water amount. The vibration data reception unit 106 may acquire the vibration data obtained by the measurements as separate pieces of vibration data for the respective measurements. The vibration feature value calculation unit 103 may calculate a vibration feature value for each measurement from vibration data for each measurement. When a plurality of vibration feature values are calculated, based on measurement results on the target soil containing a same water amount, the vibration feature value calculation unit 103 may calculate a statistical value derived from the plurality of vibration feature values as a vibration feature value of the target soil at the water amount, as described above. As described above, the statistical value is, for example, an average value, a median value, or an intermediate value.

Further, the water amount measurement unit 102 measures a water amount contained in the target soil. Then, the water amount reception unit 107 acquires the measured water amount (i.e. the measurement result of the water amount) from the water amount measurement unit 102 (Step S103). The water amount reception unit 107 transmits the received measurement result of the water amount to the soil quality determination unit 105. In Step S103, the water amount measurement unit 102 may perform two or more measurements of a water amount of the target soil containing a same water amount. The water amount reception unit 107 may acquire a plurality of measurement results of the water amount obtained by two or more measurements. In that case, the water amount reception unit 107 transmits the plurality of acquired measurement results of the water amount to the soil quality determination unit 105. Then, the soil quality determination unit 105 may calculate a statistical value (e.g., an average, an intermediate value, or a median value) of the plurality of received measurement results of the water amount as a representative value of the measured values of the water amount.

Then, for example, the water addition unit 112 increases the water amount of water contained in the target soil by adding a certain amount of water to the target soil (S104). When the water amount contained in the target soil is less than or equal to a prescribed water amount (NO in Step S105), the soil quality determination system 100 repeats the operations from Step S101 to Step S104. In other words, the vibration data reception unit 106 acquires vibration data (S101), the vibration feature value calculation unit 103 calculates a vibration feature value (S102), and the water amount reception unit 107 acquires water amount data (S103), again. Then, the water addition unit 112 adds the certain amount of water to the target soil. The soil quality determination system 100 repeats the cycle from Step S101 to Step S104 until the water amount exceeds the prescribed amount. The water amount used in the determination in Step S105 may be a water amount acquired in Step S103, the amount being measured by the water amount measurement unit 102. By repeating the operations from Step S101 to Step S105, the soil quality determination system 100 generates a damping factor-water amount distribution of the target soil.

When the water amount exceeds the prescribed water amount (YES in Step S105), the soil quality determination unit 105 selects a comparison target model from models not having been selected as comparison target models, damping factor-water amount distributions of the models being stored in the model storage unit 104 (Step S106). The comparison target model is a model to be a comparison target, that is, a model to be compared with the target soil. Then, the soil quality determination unit 105 compares a distribution of a damping factor with respect to a water amount (i.e. a damping factor-water amount distribution) of the target soil with that of the comparison target model (Step S107). In Step S107, the soil quality determination unit 105 calculates a degree of similarity of the damping factor-water amount distribution between the comparison target model and the target soil. When the comparison of damping factor-water amount distributions in Step S107 is not completed for every model a damping factor-water amount distribution of which is stored in the model storage unit 104 (NO in Step S108), the soil quality determination unit 105 repeats the operations in Step S106 and Step S107. Thus, the soil quality determination unit 105 performs selection of a comparison target model (Step S106) and comparison of damping factor-water amount distributions (Step S107) for every model stored in the model storage unit 104. When the comparison in Step S107 is completed for every model stored in the model storage unit 104 (YES in Step S108), the soil quality determination unit 105 determines a soil quality of the target soil (Step S109). In Step S109, for example, the soil quality determination unit 105 determines a soil quality model of a model with a highly ranked degree of similarity calculated in Step S107 to be a monitoring model. Specifically, for example, the soil quality determination unit 105 may determine a model with the highest similarity based on a degree of similarity to be a model representing the target soil. The soil quality determination unit 105 may determine a density of a model with the highest similarity based on a degree of similarity to be a density of the target soil.

As described above, the soil quality determination unit 105 may generate a model representing the target soil, based on scores of a plurality of models with highly ranked degrees of similarity. In that case, as described above, the soil quality determination unit 105 selects a plurality of models, based on a degree of similarity. The soil quality determination unit 105 may select a predetermined number of models in descending order of a degree of similarity. The soil quality determination unit 105 may select a model with a degree of similarity greater than a predetermined criterion. The soil quality determination unit 105 may select a model with a degree of similarity greater than a predetermined criterion, out of a predetermined number of models selected in descending order of a degree of similarity. Based on scores (i.e. degrees of similarity) of the plurality of models, the soil quality determination unit 105 determines ratios (i.e. weights) and multiplies a parameter representing a model by the ratio of the model. Selecting a plurality of models corresponds to estimating soil types of soils mixed in the target soil. Determining a ratio (i.e. weight) corresponds to determining a mixing ratio of a soil in a selected model. By adding up parameters of a plurality of models, each parameter being multiplied by a ratio, for each parameter type, the soil quality determination unit 105 generates a soil quality model representing the target soil. In this case, the soil quality determination unit 105 may further calculate a density of a soil in which the plurality of models are mixed in volumes proportional to the determined ratios to be a density of the target soil.

For example, the output unit 108 may output the determined soil quality model (e.g. a function expression representing the soil quality model or a parameter of the function expression) and density to an output device (unillustrated) such as a display.

The present example embodiment described above is able to calculate a safety factor of a slope, without previously obtaining a quality of a measurement target soil. The reason is that, based on a vibration feature value-density distribution of a measurement target soil, the soil quality determination unit 105 compares the vibration feature value-density distribution with a model soil type with a known quality, and based on the result, determines a quality of the measurement target soil. Determining a quality to be used for calculating a safety factor of a slope enables calculation of the safety factor of the slope formed of the measurement target soil.

### Second Example Embodiment

### Configuration of Second Example Embodiment

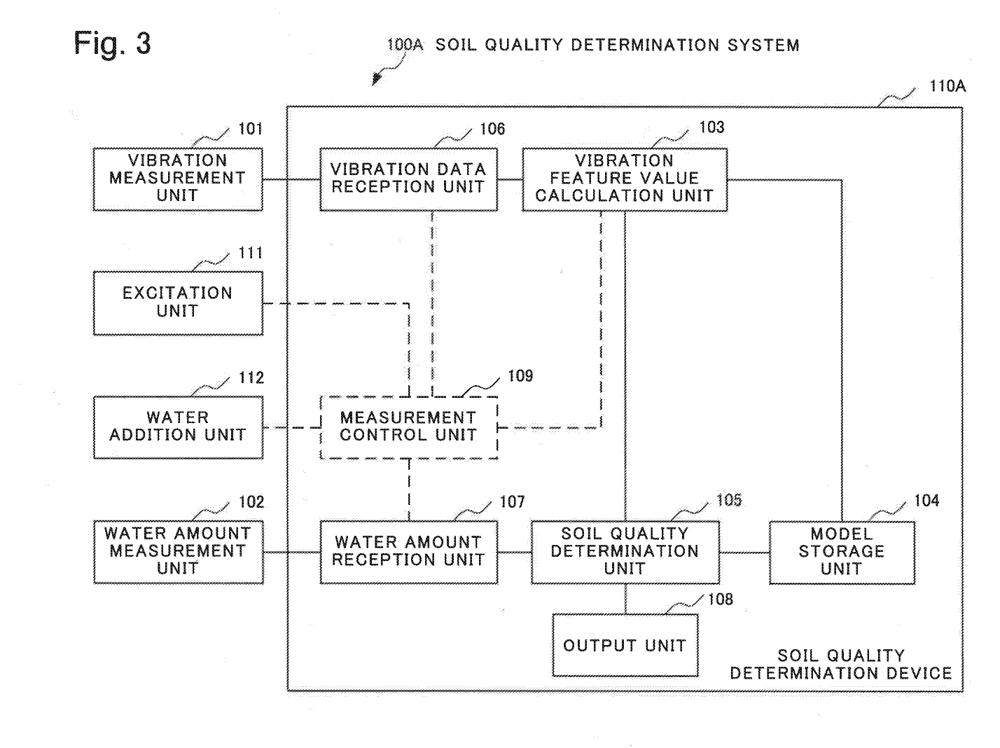

FIG. 3 is a diagram illustrating a configuration of a soil quality determination system 100A according to the present example embodiment. The soil quality determination system 100A according to the present example embodiment is identical to the soil quality determination system 100 according to the first example embodiment except for a difference described below. A part in common with the first example embodiment is omitted in the following description.

The soil quality determination system 100A according to the present example embodiment includes a soil quality determination device 110A in place of the soil quality determination device 110. A vibration feature value calculation unit 103 according to the present example embodiment may be connected to a model storage unit 104 and may read model data and the like stored in the model storage unit 104.

The model storage unit 104 according to the present example embodiment stores model data for each combination of a soil type, a density, and a pass frequency band of a frequency filter (e.g. a band-pass filter). The model data include information about a resonance frequency in addition to a function expression modeling a parameter required for a slope stability analysis formula by a vibration feature value, and a distribution of a vibration feature value with respect to a soil water amount (i.e. a vibration feature value-water amount distribution). The model data may include a parameter, such as a coefficient, specifying a function expression instead of the function expression itself.

A model according to the present example embodiment is a combination of a type of a soil and a density of the soil. Model data according to the present example embodiment include a soil quality model, and a distribution of a combination of a water amount and a vibration feature value for a plurality of different pass frequency bands. The vibration feature value is a damping factor. The distribution of a combination of a water amount and a vibration feature value is a damping factor-water amount distribution. A combination of a plurality of pass frequency bands may be the same throughout a plurality of different soil quality models. The combination of a plurality of pass frequency band may not necessarily be the same throughout the plurality of soil type models. The plurality of different pass frequency bands may be predetermined.

The pass frequency band indicates a frequency range in which signal attenuation is small, in, for example, frequency filtering by a band-pass filter or the like, to be described later. The pass frequency band may be expressed by at least one of a lower frequency limit and an upper frequency limit. For example, the lower frequency limit is a frequency indicating a lower limit of a frequency range in which signal attenuation is small. For example, the upper frequency limit is a frequency indicating an upper limit of a frequency range in which signal attenuation is small. For example, the lower frequency limit and the upper frequency limit may be frequencies at inflection points in a frequency filtering characteristic (a curve exhibiting a relation between a frequency and a passing ratio). The lower frequency limit and the upper frequency limit may respectively be a lower limit and an upper limit of a frequency range in which signal attenuation is small, the range being based on another definition. The pass frequency band may be expressed by the lower frequency limit and a frequency width. The frequency width indicates a difference between the upper frequency limit and the lower frequency limit. The pass frequency band may be expressed by the upper frequency limit and the frequency width. The pass frequency band may be expressed by a center frequency and the frequency width. The pass frequency band may include an overlap with another pass frequency band.

The vibration feature value calculation unit 103 performs frequency filtering of passing a vibration at a specific frequency band (the aforementioned pass frequency band) and attenuating vibrations at frequencies other than the frequency band on measured vibration data acquired by the vibration data reception unit 106 from the vibration measurement unit 101. Specifically, for each combination of a model and a pass frequency band, the vibration feature value calculation unit 103 may perform frequency filtering of passing a vibration at the pass frequency band and attenuating vibrations at frequencies other than the pass frequency band on measured vibration data. More specifically, the vibration feature value calculation unit 103 may select a model and read data representing a pass frequency band of the selected model from the model storage unit 104. Then, the vibration feature value calculation unit 103 may perform frequency filtering processing of passing a vibration at the pass frequency band and attenuating vibrations at frequencies other than the pass frequency band on the measured vibration data. For each model model data of which are stored in the model storage unit 104, the vibration feature value calculation unit 103 may repeat frequency filtering processing until every combination of a model and a pass frequency band is selected. The vibration feature value calculation unit 103 further calculates a vibration feature value by use of vibration data generated by performing frequency filtering by the measured vibration data.

The soil quality determination unit 105 generates a water amount-vibration feature value distribution of the target soil for each pass frequency band. Then, for each pass frequency band, the soil quality determination unit 105 compares the water amount-vibration feature value distribution of the target soil with a water amount-vibration feature value distribution of a model. Specifically, the soil quality determination unit 105 selects a water amount-vibration feature value distribution of a model, the distribution being stored in the model storage unit 104 and having a same pass frequency band in frequency filtering as that for vibration data from which the water amount-vibration feature value distribution of the target soil is derived. The soil quality determination unit 105 calculates a degree of similarity between the water amount-vibration feature value distribution of the target soil and the selected water amount-vibration feature value distribution. For each pass frequency band in frequency filtering performed on the vibration data from which the water amount-vibration feature value distribution of the target soil is derived, the soil quality determination unit 105 repeats the aforementioned selection and calculation of a degree of similarity.

The soil quality determination unit 105 may calculate a sum of the degrees of similarity calculated for the respective plurality of pass frequency bands as a degree of similarity between the target soil and the model soil type. The sum of the degrees of similarity may be a weighted sum. Specifically, the sum of the degrees of similarity in that case may be a value obtained by adding up a product of a degree of similarity at a pass frequency band and a weight based on a width of the pass frequency band for all of the plurality of pass frequency bands. The calculation method of a sum of degrees of similarity is not limited to the above.

The soil quality determination unit 105 may determine a statistical value of degrees of similarity with respect to combinations of a model soil type and pass frequency bands to be a degree of similarity of the model soil type, the statistical value including a minimum value, a maximum value, an intermediate value, a median value, or an average value.

### Operation of Second Example Embodiment

Next, an operation of the soil quality determination system 100A according to the second example embodiment of the present invention will be described in detail with reference to a drawing. Detailed description of an operation identical to that of the soil quality determination system 100 according to the first example embodiment is omitted as appropriate below.

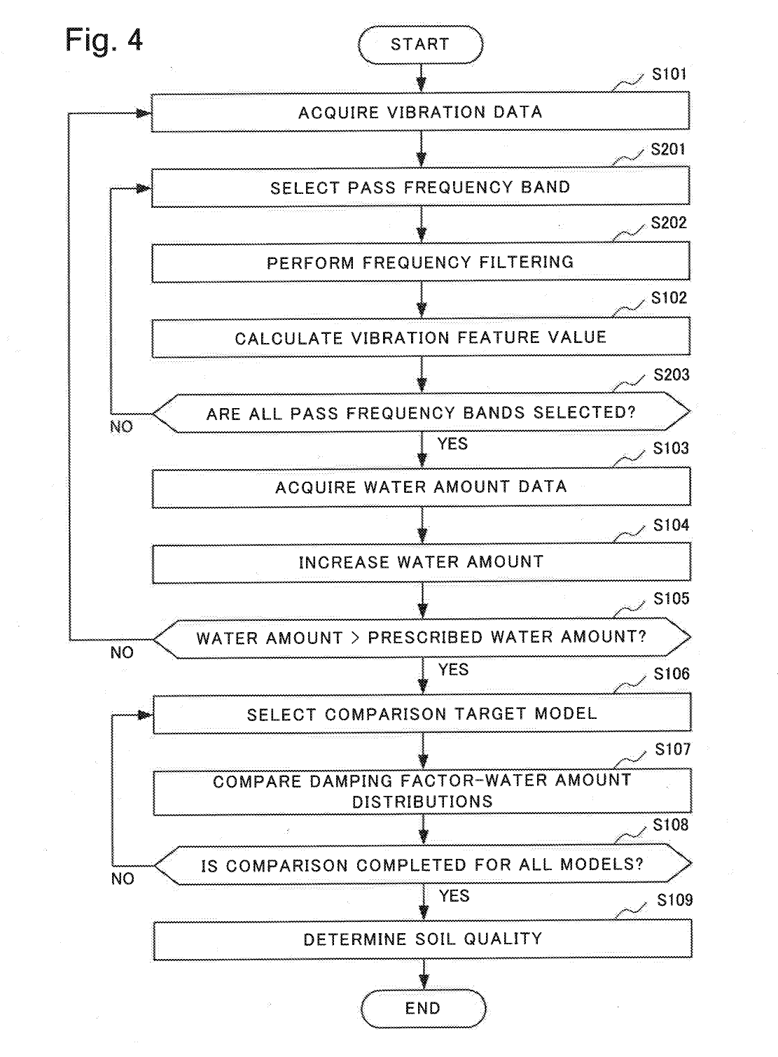

FIG. 4 is a flowchart illustrating an operation example of the soil quality determination system 100A according to the present example embodiment. First, the vibration measurement unit 101 measures vibration of a target soil. Then, the vibration data reception unit 106 receives a signal representing a measurement result of the vibration of the target soil from the vibration measurement unit 101. The vibration data reception unit 106 converts the received signal representing the vibration measurement result into vibration data in a form that can be handled by the vibration feature value calculation unit 103 (i.e. time-series data representing the measured vibration). The vibration data reception unit 106 transmits the vibration data to the vibration feature value calculation unit 103. The vibration feature value calculation unit 103 acquires the vibration data being time-series data representing the vibration measurement result from the vibration data reception unit 106 (Step S101).

Next, the vibration feature value calculation unit 103 selects an unselected pass frequency band out of the plurality of the aforementioned pass frequency bands (Step S201). The vibration feature value calculation unit 103 performs frequency filtering based on the selected pass frequency band on the vibration data, which are detected (on which sensing is performed) by the vibration measurement unit 101 and acquired in Step S101 (Step S202). The vibration feature value calculation unit 103 calculates a vibration feature value of frequency-filtered data, based on the vibration data resulting from the frequency filtering (i.e. vibration data in which signals at frequencies other than the pass frequency band are attenuated by the frequency filtering) (Step S102). When an unselected pass frequency band exists in the plurality of the aforementioned pass frequency bands (NO in Step S203), the soil quality determination system 100A repeats the operations in and after Step S201. When a combination of pass frequency bands differs for each model soil type, the vibration feature value calculation unit 103 may repeat the operations in Step S201, Step S202, and Step S102 for all the different pass frequency bands for all the model soil types. In the following description, the number of different pass frequency bands for every model soil type is also expressed as the number of filter patterns.

When all the pass frequency bands are selected (YES in Step S203), the water amount reception unit 107 acquires water amount data (Step S103). Then, for example, the water addition unit 112 increases a water amount of the target soil through control by the measurement control unit 109 (Step S104). Operations in Step S103 and Step S104 are respectively identical to the operations in Step S103 and Step S104 of the first example embodiment. When the water amount is less than or equal to a prescribed water amount (NO in Step S105), the soil quality determination system 100A repeats the operations from Step S101 to Step S105. The soil quality determination system 100A repeats similar operations until the water amount reaches the prescribed water amount. Consequently, a water amount-vibration feature value distribution of the target soil for each of the selected pass frequency band is obtained.

When the water amount exceeds the prescribed water amount (YES in Step S105), a model soil type to be compared with the target soil is selected from unselected model soil types, model data of which are stored in the model storage unit 104 (Step S106). The soil quality determination unit 105 compares distributions of a damping factor with respect to a water amount between the target soil and the model soil type (Step S107). In Step S107, the soil quality determination unit 105 may compare the water amount-vibration feature value distribution of the target soil with the water amount-vibration feature value distribution of the selected comparison target model for each of the selected pass frequency bands.

When a soil quality model for which comparison of water amount-vibration feature value distributions is not completed exists in the soil quality models, model data of which are stored in the model storage unit 104 (NO in Step S108), the soil quality determination unit 105 in the soil quality determination system 100A repeats the operations in Step S106 and Step S107. The soil quality determination unit 105 may repeat the operations in Step S106 and Step S107 until all the soil quality models model data of which are stored in the model storage unit 104 are selected.

When comparison of water amount-vibration feature value distributions for every soil quality model model data of which are stored in the model storage unit 104 is completed (YES in Step S108), the soil quality determination unit 105 determines a soil quality (Step S109). Specifically, the soil quality determination unit 105 determines a soil quality model with a degree of similarity being highly ranked in the comparison in Step S107 to be a monitoring model. Similarly to the first example embodiment, the soil quality determination unit 105 may employ a soil quality model with the highest degree of similarity as a monitoring model. The soil quality determination unit 105 may select a predetermined number of soil quality models in descending order of a degree of similarity. The soil quality determination unit 105 may determine a ratio (i.e. a weight) of a soil quality model depending on scores (degrees of similarity) of the plurality of selected soil quality models. The soil quality determination unit 105 may use a soil quality model generated by multiplying a soil quality of a selected model by a weight and adding up the soil quality models multiplied by the weights as a monitoring model. Specifically, the soil quality determination unit 105 may calculate a parameter of the monitoring model by multiplying a parameter in a function expression expressing a soil quality model of a model soil type by a weight determined for each model soil type and adding up the parameters multiplied by the weights.

The present example embodiment provides the same effect as that provided by the first example embodiment. The reason is the same as the reason the effect according to the first example embodiment is provided.

The present example embodiment further provides an effect of improving a system of soil quality determination. The reason is that the vibration feature value calculation unit 103 performs frequency filtering processing using a plurality of different pass frequency bands. Then, the soil quality determination unit 105 determines a soil quality model of a target soil by using, for comparison, water amount-vibration feature value distributions generated for the respective pass frequency bands.

### Third Example Embodiment

### Configuration of Third Example Embodiment

Next, a third example embodiment of the present invention will be described in detail with reference to drawings.

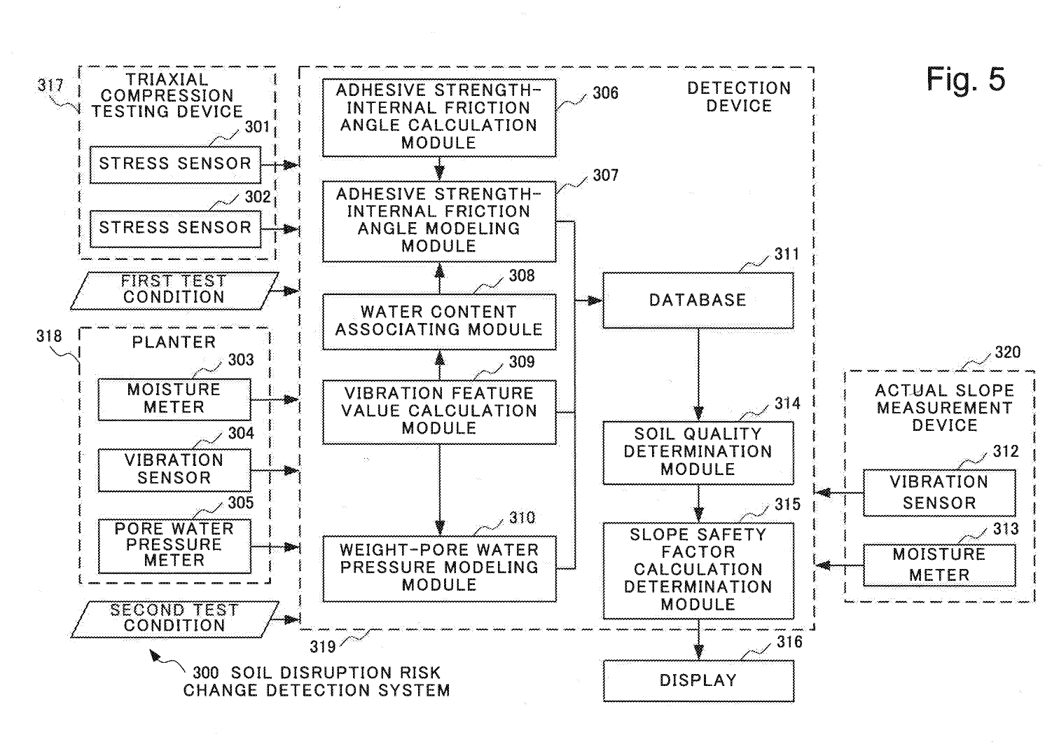

FIG. 5 is a block diagram illustrating a configuration example of a soil disruption risk change detection system 300 according to the present example embodiment. The soil disruption risk change detection system 300 includes the functions of the soil quality determination system according to the first or second example embodiment. For example, the soil quality determination system according to any one of the aforementioned example embodiments corresponds to a database 311, a soil quality determination module 314, and an actual slope measurement device 320, to be described later. In other words, the database 311, the soil quality determination module 314, and the actual slope measurement device 320 operate as the soil quality determination system according to the first or second example embodiment. In the following description, the soil disruption risk change detection system 300 is abbreviated to a “detection system 300.”

Referring to FIG. 5, the detection system 300 includes a triaxial compression testing device 317, a planter 318, a detection device 319, a display 316, and an actual slope measurement device 320. The detection device 319 is communicably connected to the triaxial compression testing device 317, the planter 318, the display 316, and the actual slope measurement device 320. For example, the detection device 319 is further communicably connected to a terminal device (unillustrated) for inputting a first test condition and a second test condition to the detection device 319.

The triaxial compression testing device 317 includes a stress sensor 301 and a stress sensor 302.

The planter 318 includes a moisture meter 303, a vibration sensor 304, and a pore water pressure meter 305.

The detection device 319 includes an adhesive strength-internal friction angle calculation module 306, an adhesive strength-internal friction angle modeling module 307, and a water content associating module 308. The detection device 319 further includes a vibration feature value calculation module 309 and a weight-pore water pressure modeling module 310. The detection device 319 further includes the database 311, the soil quality determination module 314, and a slope safety factor calculation determination module 315. The detection device 319 may be provided by a single device. The detection device 319 may be provided by a plurality of devices each including at least any one of the modules and the database 311 that are included in the detection device 319.

The actual slope measurement device 320 includes a vibration sensor 312 and a moisture meter 313. The vibration sensor 312 and the moisture meter 313 are both buried at one point on a slope at a depth of, for example, 10 centimeters (cm).

The devices included in the detection system 300 roughly operate as follows.

The triaxial compression testing device 317 performs a test for calculating an adhesive strength and an internal friction angle.

The planter 318 acquires data for modeling a clod weight and a volume water content.

The detection device 319 models an adhesive strength, an internal friction angle, a clod weight, and a pore water pressure that are used in a slope stability analysis formula by the modified Fellenius method from data obtained through a test using the triaxial compression testing device 317 and the planter 318. The detection device 319 further stores model data in the database 311 for each soil type and density. The detection device 319 further determines a soil type and a density of an actual slope from actual slope data, and based on the determination result, determines a suitable model from the database 311. Based on the selected model, the detection device 319 further calculates a safety factor of the slope, based on the actual slope data. The detection device 319 further estimates a state change, based on the calculated safety factor, and changes a display content displayed on the display 316 depending on the estimated state change.

The display 316 displays a display content depending on an estimated state change.

Each component in each device included in the detection system 300 will be described in more detail below.

The stress sensor 301 and the stress sensor 302 measure a shearing stress of a clod being set on the triaxial compression testing device 317 and being compressed.

The moisture meter 303 measures a water amount of a clod being set on the planter 318, and undergoing water addition and excitation, a soil type, a density, and a water content being set to the clod.

The vibration sensor 304 measures vibration of the aforementioned clod set on the planter 318.

The pore water pressure meter 305 measures a pore water pressure of the aforementioned clod set on the planter 318.

The planter 318 further measures a weight of the aforementioned clod with an unillustrated weighing scale.

The adhesive strength-internal friction angle calculation module 306 calculates an adhesive strength and an internal friction angle, based on data by a triaxial compression test performed based on a first test condition set to variously change each of a soil type, a degree of compaction, and a water content.

The vibration feature value calculation module 309 calculates a vibration feature value, based on data by a water addition excitation test performed by use of the planter 318, based on a second test condition similarly set to variously change a soil type, a degree of compaction, and a water content.

The water content associating module 308 associates a water content with a water amount and a vibration feature value.

Using a water content as a key, the adhesive strength-internal friction angle modeling module 307 models an adhesive strength and an internal friction angle by a water amount and a vibration feature value. For example, the adhesive strength-internal friction angle modeling module 307 specifies a relational expression expressing a relation between each of an adhesive strength and an internal friction angle, and a water amount and a vibration feature value.

The weight-pore water pressure modeling module 310 models a weight and a pore water pressure by a damping factor. For example, the weight-pore water pressure modeling module 310 specifies a relational expression expressing a relation between each of a weight and a pore water pressure, and a damping factor.

The database 311 stores model functions of an adhesive strength, an internal friction angle, a weight, and a pore water pressure, and distribution data of a vibration feature value with respect to a water content, for each soil type and density. For example, the database 311 is a storage device operating as a model storage unit 104. The database 311 may store the model functions and the distribution data in a form of a database and may be an information processing device performing input and output of the model functions and the distribution data.

The soil quality determination module 314 selects a model used for safety monitoring of an actual slope from the database 311, based on vibration data and a water amount that are measured at the actual slope.

The slope safety factor calculation determination module 315 calculates a safety factor of a slope by use of a model a condition of which matches a determined soil type and a determined density, and based on the calculated safety factor, determines a degree of safety.

The vibration sensor 312 measures vibration of a slope.

The moisture meter 313 measures a water amount of a slope.

### Operation of Third Example Embodiment

Next, an operation of the detection system 300 according to the present example embodiment will be described in detail with reference to drawings.

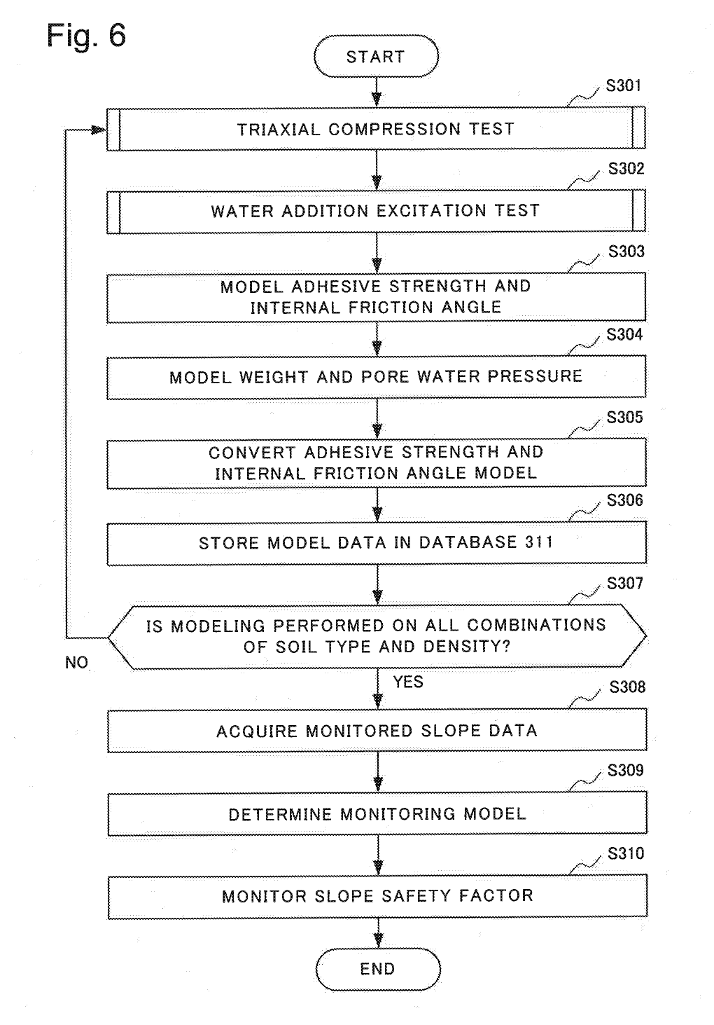

FIG. 6 is a flowchart illustrating an operation example of the detection system 300 according to the present example embodiment. When the operation illustrated in FIG. 6 is started, a combination of a soil type and a density that are modeled first may be selected.

First, the detection system 300 performs a triaxial compression test by the triaxial compression testing device 317, based on a test condition (the first test condition in the example illustrated in FIG. 5) (Step S301). Specifically, the detection system 300 selects a soil type and a density (i.e. a model soil type) set as the first test condition. By use of a clod composed of a soil of the model soil type, the detection system 300 performs triaxial compression tests by the triaxial compression testing device 317 in a plurality of water content patterns. The triaxial compression testing device 317 transmits an adhesive strength, an internal friction angle, and the like that are obtained as a result of the triaxial compression tests to the detection device 319. The triaxial compression test will be described in detail later.

The detection system 300 further performs a water addition excitation test by the planter 318 in accordance with a test condition (the second test condition in the example illustrated in FIG. 5) specifying a soil type and a density (Step S302). The planter 318 transmits a clod weight, a pore water pressure, vibration data, and the like that are obtained by the water addition excitation test to the detection device 319 for, for example, each water content at which the test is performed. The water addition excitation test will be described in detail later.

Next, the detection system 300 models the adhesive strength, the internal friction angle, the clod weight, and the pore water pressure that are obtained from the triaxial compression test and the water addition excitation test by a damping factor and a water amount. In other words, the detection system 300 specifies relational expressions (e.g. parameters in relational expressions in a predetermined form) expressing relations between each of the adhesive strength, the internal friction angle, the clod weight, and the pore water pressure, and a damping factor and a water amount.

Specifically, the adhesive strength-internal friction angle modeling module 307 in the detection device 319 models the adhesive strength and the internal friction angle that are obtained by the performed triaxial compression test as a function of a water content (Step S303). As will be described later, the adhesive strength and the internal friction angle are calculated by the adhesive strength-internal friction angle calculation module in the detection device 319 in a triaxial compression test.

The weight-pore water pressure modeling module 310 in the detection device 319 models the clod weight and the pore water pressure obtained by the performed water addition excitation test by a vibration feature value of vibration data acquired at the same time when the clod weight and the pore water pressure are obtained (Step S304). The weight-pore water pressure modeling module 310 stores in the database 311 models of the clod weight and the pore water pressure that are modeled by the vibration feature value.

The adhesive strength-internal friction angle modeling module 307 in the detection device 319 further converts the adhesive strength and the internal friction angle model into a model based on the vibration feature value (Step S305). Specifically, the adhesive strength-internal friction angle modeling module 307 models the adhesive strength and the internal friction angle by the vibration feature value by associating the adhesive strength and the internal friction angle with a vibration feature value at each water content, with a water content as a key. By modeling the adhesive strength and the internal friction angle by the vibration feature value, the adhesive strength-internal friction angle modeling module 307 derives, for example, the aforementioned relational expression or a parameter capable of specifying the relational expression, as a soil quality model.

The detection device 319 (the adhesive strength-internal friction angle modeling module 307 in the detection device 319) stores data on the models (model data) obtained in or before Step S305 in the database 311 (Step S306). The detection device 319 may add the obtained model data to the database 311 stored in the model storage unit 104.

When a combination on which modeling is not performed is included in combinations of a soil type and a density (NO in Step S307), the detection system 300 selects the combination of the soil type and the density on which modeling is not performed. Then, the detection system 300 repeats the operations from Step S301 with respect to the selected combination of the soil type and the density.

When modeling is performed on every combination of a soil type and a density (YES in Step S307), the detection system 300 ends model generation by tests and starts monitoring of a slope.

The soil quality determination module 314 in the detection device 319 acquires data of the vibration sensor 312 and the moisture meter 313 at a monitoring target slope (Step S308). The soil quality determination module 314 determines a monitoring model, based on the obtained data (Step S309). The slope safety factor calculation determination module 315 calculates a slope safety factor by use of the model determined in Step S309 and monitors safety of the slope by monitoring the calculated slope safety factor (Step S310). The operations from Step S308 to Step S310 will be described in detail later.

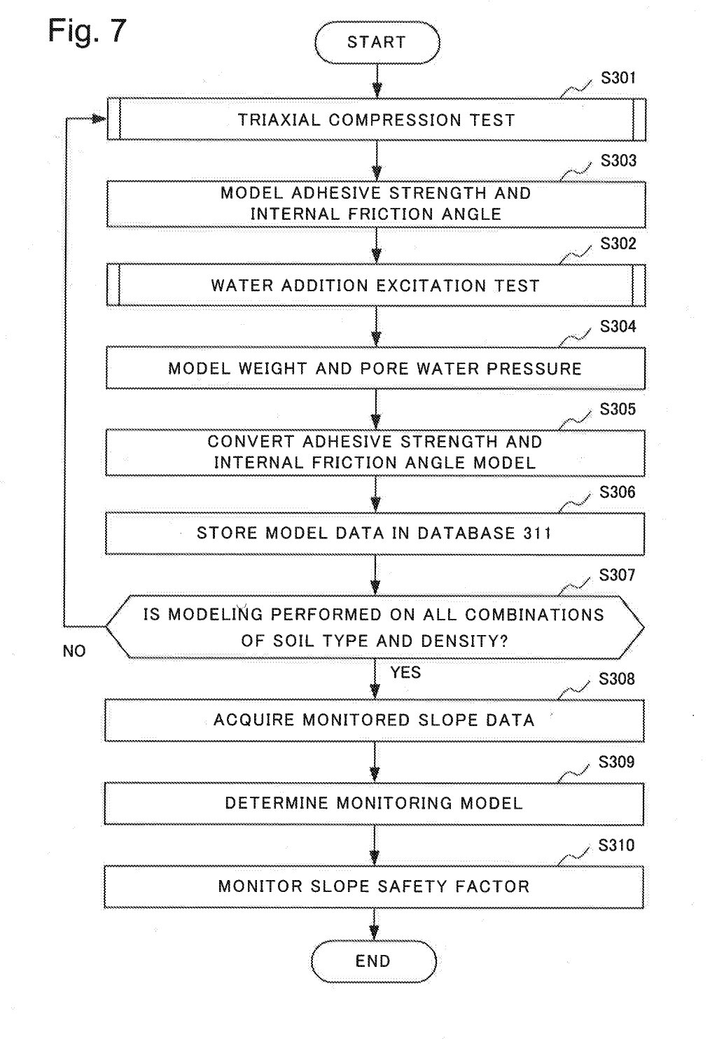

FIG. 7 is a flowchart illustrating another operation example of the detection system 300 according to the present example embodiment. Comparing the flowchart illustrated in FIG. 7 with the flowchart illustrated in FIG. 6, an operation in Step S303 is performed subsequently to an operation in Step S301 in the example illustrated in FIG. 7. Then, an operation in Step S302 is performed after the operation in Step S303. Operations in and after Step S304 are performed after the operation in Step S302. The operation illustrated in FIG. 7 is identical to the operation illustrated in FIG. 6 except for the difference described above.

Next, an operation of a triaxial compression test by the detection system 300 according to the present example embodiment will be described in detail with reference to a drawing.

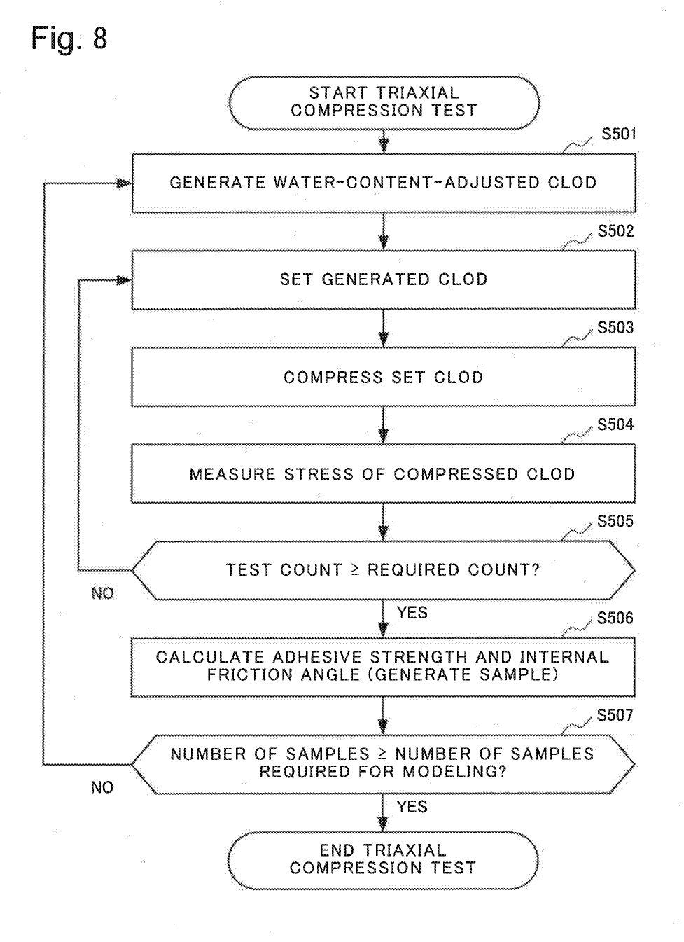

FIG. 8 is a flowchart illustrating an operation example of a triaxial compression test by the detection system 300 according to the present example embodiment. The flowchart in FIG. 8 includes an operation of an operator who operates the triaxial compression testing device 317 in the triaxial compression test.

First, the operator prepares a water-content-adjusted clod being a clod a water content of which is adjusted in accordance with a test condition (Step S501). Then, the operator sets the generated clod on the triaxial compression testing device 317 (Step S502).

For example, the triaxial compression testing device 317 compresses the set clod in accordance with an instruction by the operator (Step S503). The triaxial compression testing device 317 measures a shearing stress of the set clod (Step S504). When a test count is less than a required count being a test count required for calculation of an adhesive strength and an internal friction angle (NO in Step S505), the triaxial compression testing device 317 repeats the operations from Step S502 to Step S504. The test count is the number of times a test represented by the operations from Step S502 to Step S504 is performed. When the test count is greater than or equal to the required count (YES in Step S505), the detection device 319 calculates an adhesive strength and an internal friction angle by use of data obtained by repeating the test (Step S506). In the description of FIG. 8, the adhesive strength and the internal friction angle calculated in each operation in Step S506 are expressed as a sample. When the number of generated samples is less than the number of samples required for modeling (NO in Step S507), the operator and the triaxial compression testing device 317 repeat the operation from Step S501 to Step S506. When the number of generated samples is greater than or equal to the number of samples required for modeling (YES in Step S507), the operation of the triaxial compression test ends.

Next, an operation of processing in a water addition excitation test by the detection system 300 according to the present example embodiment will be described in detail with reference to a drawing.

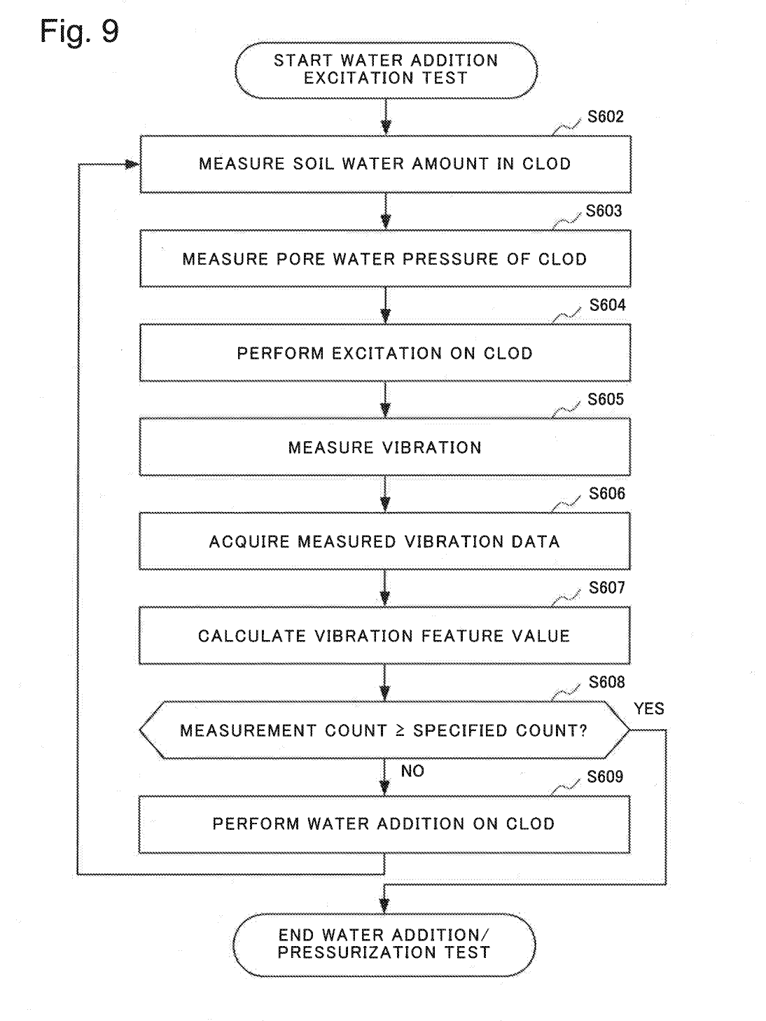

FIG. 9 is a flowchart illustrating an operation example of the processing in a water addition excitation test by the detection system 300 according to the present example embodiment.

When the processing of a water addition excitation test illustrated in FIG. 9 is started, for example, a clod is set on the planter 318 by an operator operating the detection system 300. In the following description, operations from Step S602 to Step S607 represent a test. A test count refers to the number of times the operations from Step S602 to Step S607 are performed.

First, the moisture meter 303 in the planter 318 measures a soil water amount being an amount of water contained in the set clod (Step S602). The moisture meter 303 transmits the measured soil water amount to the detection device 319. Water addition is not performed on the set clod in the first water amount measurement. In other words, water addition is not performed on the set clod in the first test.

Next, the pore water pressure meter 305 measures a pore water pressure of the clod (Step S603). The pore water pressure meter 305 transmits the measured pore water pressure to the detection device 319.

Next, for example, the operator performs excitation being application of vibration to the clod by a vibration generator (unillustrated) for applying vibration to a clod, the vibration generator being installed on the planter 318 (Step S604). The vibration generator may perform excitation on the clod in accordance with control by the detection device 319 or a terminal device (unillustrated).

The vibration sensor 304 measures vibration of the clod on which excitation is being performed (Step S605). The vibration sensor 304 transmits vibration data obtained by the vibration measurement to the detection device 319.

The vibration feature value calculation module 309 in the detection device 319 acquires from the vibration sensor 304 the vibration data obtained by the vibration sensor 304 measuring the vibration of the clod (Step S606).

Next, the vibration feature value calculation module 309 in the detection device 319 calculates a vibration feature value by use of the obtained vibration data (Step S607). For example, the vibration feature value calculation module 309 calculates a resonance frequency or a damping factor as the vibration feature value.

When a measurement count is less than a specified count (NO in Step S608), the aforementioned operator performs water addition being an operation of adding a predetermined amount of water to the clod (S609). The measurement count is the number of times a test represented by the operations from Step S602 to Step S607 is performed. The specified count is the number of times specified as the number of times the test represented by the operations from Step S602 to Step S607 is to be performed. A water addition device adding a specified amount of water to the clod may be installed on the planter 318. Then, the water addition device may perform water addition. An amount of water added to the clod is determined by a water content specified by a test condition (the second test condition in the example in FIG. 5). For example, the water content associating module 308 in the detection device 319 may calculate an amount of water to be added to the clod, based on the second test condition, and notify the calculated amount of water to the aforementioned operator by, for example, an image and voice. The aforementioned operator may calculate an amount of water in accordance with the test condition. For example, the water content associating module 308 in the detection device 319 may instruct the water addition device on an amount of water to be added to the clod per addition.

When water addition to the clod is performed, the operation of the detection system 300 returns to the operation in Step S602. Then, the detection system 300 performs a next round of the test. The detection system 300 repeats the operations (i.e. test) from Step S602 to Step S607.

When the test count is greater than or equal to the specified count (YES in Step S608), the detection system 300 ends the operation illustrated in FIG. 9.

Through the operations in FIGS. 7, 8, and 9 described above, the detection system 300 derives, for each soil type and density, models of an adhesive strength, an internal friction angle, a clod weight, and a pore water pressure based on a vibration feature value, and a distribution of a vibration feature value with respect to a change in a water content when water is added. As described above, the detection system 300 stores the derived models and distributions in the database 311.

Next, a monitoring operation of the detection system 300 according to the present example embodiment will be described in detail with reference to FIG. 6.

By the vibration sensor 312 and the moisture meter 313 that are installed on a slope being a monitoring target (hereinafter expressed as a monitoring target slope), the actual slope measurement device 320 measures data on vibration and a water amount of the monitoring target slope (S308). The actual slope measurement device 320 transmits the data obtained by the measurement to the detection device 319.

The soil quality determination module 314 in the detection device 319 receives the aforementioned data from the actual slope measurement device 320. Based on the received data, the soil quality determination module 314 determines a monitoring model being a soil quality model indicating a quality of a soil in the monitoring target slope (S309). The method of determining a monitoring model by the soil quality determination module 314 may be the same as the method of determining a monitoring model by the soil quality determination device 110 according to the first example embodiment. In other words, the soil quality determination module 314 may operate as the vibration feature value calculation unit 103 and the soil quality determination unit 105 of the first example embodiment. The soil quality determination module 314 may operate as the vibration feature value calculation unit 103 and the soil quality determination unit 105 of the second example embodiment.

The slope safety factor calculation determination module 315 in the detection device 319 calculates a safety factor of the monitoring target slope (also expressed as a slope safety factor) by use of the determined monitoring model. The slope safety factor calculation determination module 315 displays the calculated safety factor on the display 316 (S310). For example, the operator monitors the monitoring target slope by monitoring a display content indicating the slope safety factor displayed on the display 316 (S310). The operator uses the display content displayed on the display 316 for monitoring of the monitoring target slope. The slope safety factor calculation determination module 315 may determine whether or not the calculated safety factor indicates a higher risk than a predetermined criterion, by monitoring the calculated safety factor. When the calculated safety factor indicates a higher risk than the predetermined criterion, the slope safety factor calculation determination module 315 may display a risk on the display 316. The slope safety factor calculation determination module 315 may output voice expressing a risk with a speaker (unillustrated).

When the detection system 300 operates as the soil quality determination device 110A according to the second example embodiment, for example, the vibration feature value calculation module 309 performs frequency filtering on vibration data for a plurality of pass frequency bands in Step S606. The vibration feature value calculation module 309 extracts a vibration feature value from the frequency filtering result. In other words, the vibration feature value calculation module 309 extracts a vibration feature value for each pass frequency band. The adhesive strength-internal friction angle calculation module calculates a model for each pass frequency band and stores the calculated model in the database 311. The weight-pore water pressure modeling module calculates a model for each pass frequency band and stores the calculated model in the database. Accordingly, the database 311 stores, for each combination of a soil type, a density, and a filtering region (i.e. a pass frequency band), model functions of an adhesive strength, an internal friction angle, a weight, and a pore water pressure, a distribution of vibration feature value calculation with respect to a change in a water amount, and a resonance frequency.

The slope safety factor calculation determination module 315 sets a coefficient of a soil quality model of a soil type, based on a ratio derived by the soil quality determination module 314, generates the soil quality model of the estimated soil type, based on the set coefficient, and uses the generated soil quality model for monitoring. The slope safety factor calculation determination module 315 converts time-series data resulting from a measurement by the vibration sensor into a vibration feature value. Then, the slope safety factor calculation determination module 315 sequentially displays the adhesive strength, the internal friction angle, the clod weight, and the pore water pressure that are modeled by the vibration feature value, and a safety factor calculated by use thereof on the display 316 as a state of the monitoring target slope.

### Fourth Example Embodiment

### Configuration of Fourth Example Embodiment

Next, a fourth example embodiment of the present invention will be described in detail with reference to drawings.

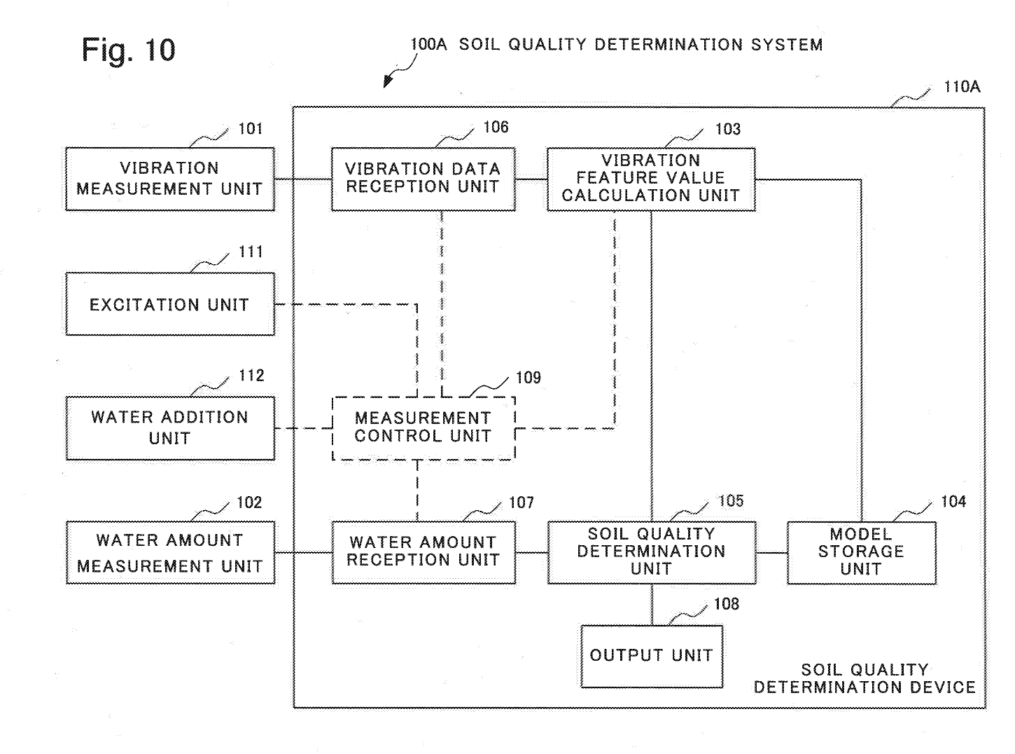

FIG. 10 is a diagram illustrating a configuration example of a soil quality determination device 110A according to the present example embodiment.

As illustrated in FIG. 10, the soil quality determination device 110A according to the present example embodiment includes a vibration measurement unit 101, a water amount measurement unit 102, a vibration feature value calculation unit 103, a model storage unit 104, and a soil quality determination unit 105. The vibration measurement unit 101 and the water amount measurement unit 102 may be communicably connected to the soil quality determination device 110A including the vibration feature value calculation unit 103, the model storage unit 104, and the soil quality determination unit 105. The soil quality determination device 110A further includes a vibration data reception unit 106, a water amount reception unit 107, and an output unit 108. The soil quality determination device 110A may further include a measurement control unit 109.

Similarly to the model storage unit 104 according to the second example embodiment, the model storage unit 104 according to the present example embodiment stores data for each pass frequency band used when a soil type, a density, and a frequency feature value are calculated. The model storage unit 104 stores a resonance frequency, in addition to a function expression modeling a parameter required for each slope stability analysis formula by a damping factor, and a distribution of the damping factor with respect to a soil water amount.

The soil quality determination device 110A according to the present example embodiment is identical to the soil quality determination device 110A according to the second example embodiment except for the following difference. An operation of comparing damping factor-water amount distributions by the soil quality determination unit 105 in the soil quality determination device 110A according to the present example embodiment is different from the operation of comparing damping factor-water amount distributions by the soil quality determination unit 105 in the soil quality determination device 110A according to the second example embodiment.

### Operation of Fourth Example Embodiment

Next, an operation of the soil quality determination device 110A according to the fourth example embodiment will be described in detail with reference to drawings.

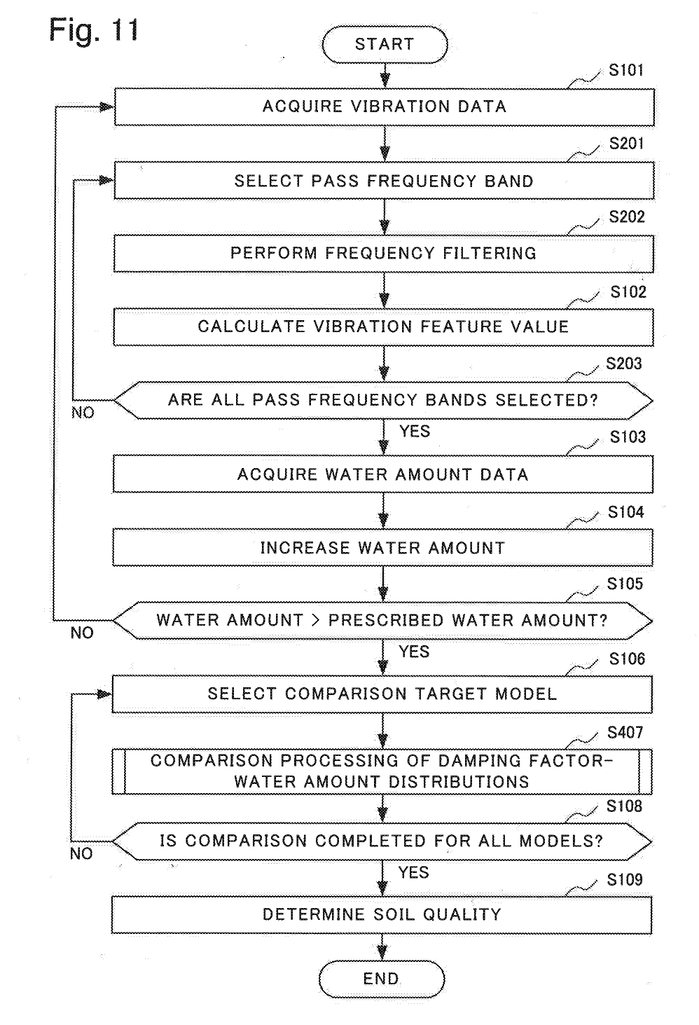

FIG. 11 is a diagram illustrating an overall operation of the soil quality determination device 110A according to the fourth example embodiment. The operation illustrated in FIG. 11 is identical to the operation of the soil quality determination device 110A according to the second example embodiment illustrated in FIG. 4, except for an operation in Step S407 next to Step S106. The difference between the operation of the soil quality determination device 110A according to the present example embodiment and the operation of the soil quality determination device 110A according to the second example embodiment will be mainly described below.

The vibration data reception unit 106 acquires time-series data detected (on which sensing is performed) by the vibration measurement unit 101 (Step S101). The vibration data reception unit 106 may receive vibration data representing the measured vibration from the vibration measurement unit 101.

For example, the vibration feature value calculation unit 103 selects an unselected pass frequency band from a plurality of predetermined pass frequency bands (Step S201). The vibration feature value calculation unit 103 performs frequency filtering passing a signal in the selected pass frequency band on the obtained vibration data (Step S202). The vibration feature value calculation unit 103 calculates a vibration feature value from the frequency-filtered vibration data (Step S102).

When an unselected pass frequency band exists (NO in Step S203), the operation of the soil quality determination device 110A returns to Step S201. Then, change of a pass frequency band (Step S201), performing frequency filtering (Step S202), and calculation of a vibration feature value (Step S102) are repeated a predetermined number of times (e.g. until all of the aforementioned plurality of frequency bands are selected). The vibration feature value according to the present example embodiment is also a damping factor. Models (e.g. the aforementioned function expression) and damping factor-water amount distributions stored in the model storage unit 104 may also be derived for each of the same plurality of frequency bands.

Next, the water amount reception unit 107 acquires water amount data representing a water amount measured by the water amount measurement unit 102 (Step S103). The water amount reception unit 107 may receive the water amount data from the water amount measurement unit 102. The soil quality determination unit 105 updates a vibration feature value-water amount distribution (a damping factor-water amount distribution in the case of the present example embodiment) for each pass frequency band, based on the calculated vibration feature value (a damping factor in the case of the present example embodiment) for each pass frequency band and the acquired water amount data. For example, the soil quality determination unit 105 may add the calculated damping factor value at a water amount represented by the acquired water amount data to the damping factor-water amount distribution data for each pass frequency band.

Then, for example, an operator of the soil quality determination device 110A increases the water amount by adding a predetermined amount of water to the target soil (Step S104). When the water amount does not reach a prescribed water amount (NO in Step S105), the operation of the soil quality determination device 110A returns to Step S101. Then, the soil quality determination device 110A repeats a similar operation until the water amount reaches the prescribed water amount (YES in Step S105). For example, the water amount used in the determination in Step S105 may be a water amount indicated by the obtained water amount data. For example, the water amount used in the determination in Step S105 may be a sum of water amounts added in Step S104. Consequently, a damping factor-water amount distribution of the measurement target soil is obtained for each pass frequency band.

The soil quality determination device 110A may repeat the operations from Step S101 to Step S203 a plurality of number of times in a state where the water amount is the same. The vibration feature value calculation unit 103 may calculate a statistical value (e.g. an average value, a median value, or an intermediate value) of a plurality of vibration feature values calculated at the same water amount. The soil quality determination unit 105 may generate a damping factor-water amount distribution by use of the vibration feature values (damping factors in the case of the present example embodiment as described above). The soil quality determination unit 105 may generate a plurality of damping factor-water amount distributions at a same pass frequency band, based on a plurality of vibration feature values obtained in a state where the water amount is the same.

When the water amount exceeds the prescribed water amount (YES in Step S105), the soil quality determination unit 105 selects a comparison target model (Step S106). The soil quality determination unit 105 selects a comparison target model being a model compared with the measurement target soil, from the models stored in the model storage unit 104 (Step S106).

As described above, according to the present example embodiment, a combination of pass frequency bands in frequency filtering applied to vibration data from which models and distributions that are stored in the model storage unit 104 are derived is identical to the combination of pass frequency bands in Step S202. In that case, the soil quality determination unit 105 may select an unselected model from all the models stored in the model storage unit 104.

A model and a distribution with a different combination of pass frequency bands in frequency filtering applied to vibration data used for the derivation may coexist with the models and the distributions that are stored in the model storage unit 104. In that case, the soil quality determination unit 105 selects an unselected model as a comparison target model from models used in frequency filtering in which the same combination as the combination of pass frequency bands in Step S202 is applied to vibration data.

Next, the soil quality determination unit 105 performs comparison processing of comparing damping factor-water amount distributions (distributions of a damping factor with respect to a water amount) between the measurement target soil and the selected model (Step S407). When the comparison is not completed for at least one of the models being selection targets, the models being stored in the model storage unit 104 (NO in Step S108), the operation of the soil quality determination device 110A returns to Step S106. Then, the soil quality determination unit 105 repeats the selection of a comparison target model in Step S106 and the comparison of damping factor-water amount distributions in Step S407.

Thus, the soil quality determination unit 105 performs the selection of a comparison target model (Step S106) and the comparison of damping factor-water amount distributions (Step S407) on every model selectable as a comparison target, the model being stored in the model storage unit 104. The soil quality determination unit 105 calculates a degree of similarity between the measurement target soil and each model being a comparison target as a result of Step S407.

When the comparison of damping factor-water amount distributions is completed for all the models selectable as comparison targets (YES in Step S108), the soil quality determination unit 105 determines a soil quality (Step S109). Specifically, similarly to the first and second example embodiments, the soil quality determination unit 105 determines a model with a highly-ranked calculated degree of similarity to be a monitoring model. The soil quality determination unit 105 may employ a soil quality model with the highest degree of similarity. The soil quality determination unit 105 may determine weights of a plurality of models with a highly-ranked degree of similarity, based on scores, multiply, by the weight, a parameter representing a soil quality model of a model for which a weight is determined, and generate a monitoring model by adding up the parameters multiplied by the weights for respective parameter types.

Next, an operation of the comparison processing of damping factor-water amount distributions in Step S407 by the soil quality determination device 110A according to the present example embodiment will be described in detail by use of drawings.

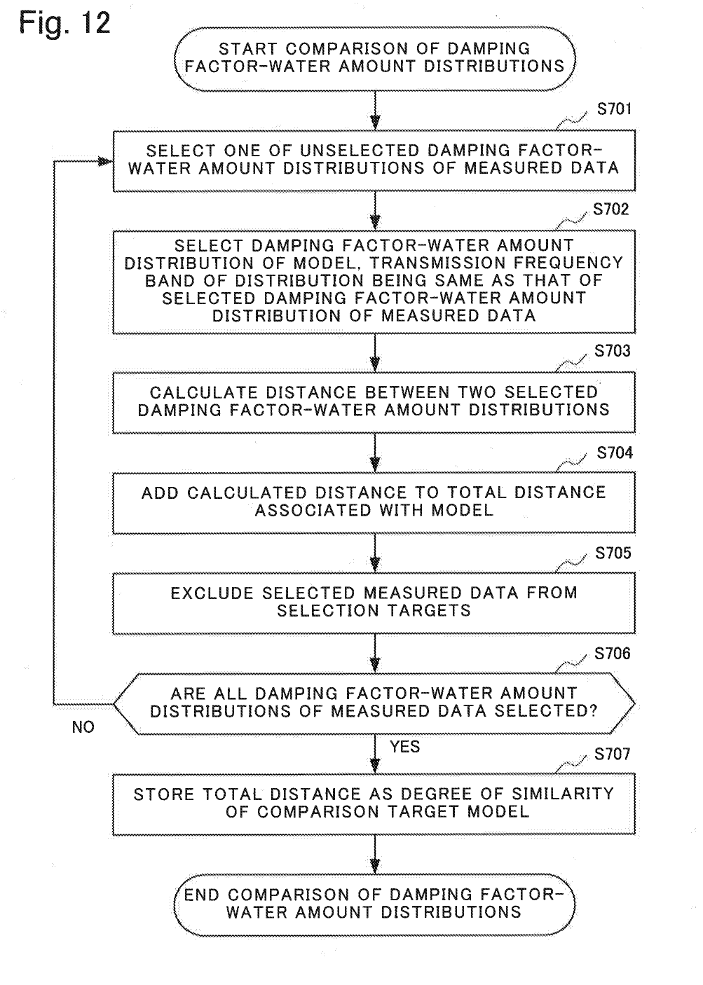

FIG. 12 is a flowchart illustrating an operation example of the comparison processing of damping factor-water amount distributions by the soil quality determination device 110A according to the present example embodiment. At the start of the operation illustrated in FIG. 12, a comparison target model to be compared with measured data is selected (Step S106 illustrated in FIG. 11).

First, an unselected damping factor-water amount distribution is selected from damping factor-water amount distributions generated based on the data measured in the operations up to Step S105 illustrated in FIG. 11 (Step S701). A damping factor-water amount distribution generated based on the data measured in the operations from Step S101 to Step S105 illustrated in FIG. 11 is also expressed as a damping factor-water amount distribution of the measured data. As described above, each damping factor-water amount distribution of the measured data is generated based on vibration data on which frequency filtering with one of the pass frequency bands is performed. In the following description, a pass frequency band used in frequency filtering performed on vibration data for which a damping factor-water amount distribution is generated is expressed as a pass frequency band of the damping factor-water amount distribution. The “unselected damping factor-water amount distribution of the measured data” in Step S701 represents an unselected damping factor-water amount distribution of the measured data in the operation illustrated in FIG. 12 with respect to the comparison target model selected in Step S106 illustrated in FIG. 11.

The soil quality determination unit 105 selects a damping factor-water amount distribution a pass frequency band of which is the same as a pass frequency band of the selected damping factor-water amount distribution of the measured data, out of damping factor-water amount distributions of comparison target models (Step S702).

The soil quality determination unit 105 calculates a distance between the two selected damping factor-water amount distributions (Step S703). One of the two selected water amount-damping factor distributions is the damping factor-water amount distribution of the measured data selected in Step S701. The other of the two selected damping factor-water amount distributions is the damping factor-water amount distribution of the comparison target model selected in Step S702. For example, the soil quality determination unit 105 may calculate an average of absolute values of differences between damping factors at same water amounts as the distance between the two damping factor-water amount distributions. For example, the soil quality determination unit 105 may calculate a root mean square of differences between damping factors at same water amounts as the distance between the two damping factor-water amount distributions. The soil quality determination unit 105 may calculate another type of distance as the distance between the two damping factor-water amount distributions.

The soil quality determination unit 105 adds the calculated distance to a total distance associated with the comparison target model (Step S704). The total distance associated with the comparison target model may be set to zero at the start of the operation illustrated in FIG. 12.

The soil quality determination unit 105 excludes a damping factor-water amount distribution of the measured data selected in Step S701 from selection targets in the next execution of Step S701 (Step S705). When an unselected damping factor-water amount distribution, that is, a damping factor-water amount distribution not excluded from the selection targets exists in the damping factor-water amount distributions of the measured data (NO in Step S706), the operation of the soil quality determination device 110A returns to Step S701. Then, the soil quality determination device 110A performs the operations from Step S701 again.

When every damping factor-water amount distribution of the measured data is selected, that is, a damping factor-water amount distribution not excluded from the selection targets does not exist (YES in Step S706), the soil quality determination unit 105 associates the calculated total distance to the comparison target model. Then, the soil quality determination unit 105 stores the total distance associated with the comparison target model as a degree of similarity of the comparison target model (Step S707). In this case, as a degree of similarity (i.e. a total distance) of a comparison target model becomes smaller, the damping factor-water amount distribution of the comparison target model becomes more similar to the damping factor-water amount distribution of the measured data. The above concludes the operation illustrated in FIG. 12. When storing total distances associated with comparison target models, the soil quality determination unit 105 may sort the total distances in ascending order. When storing a total distance associated with a comparison target model, the soil quality determination unit 105 may assign a rank of shortness to the total distance. The soil quality determination unit 105 may store a total distance associated with a comparison target model as a degree of similarity of the comparison target model in, for example, the model storage unit 104.

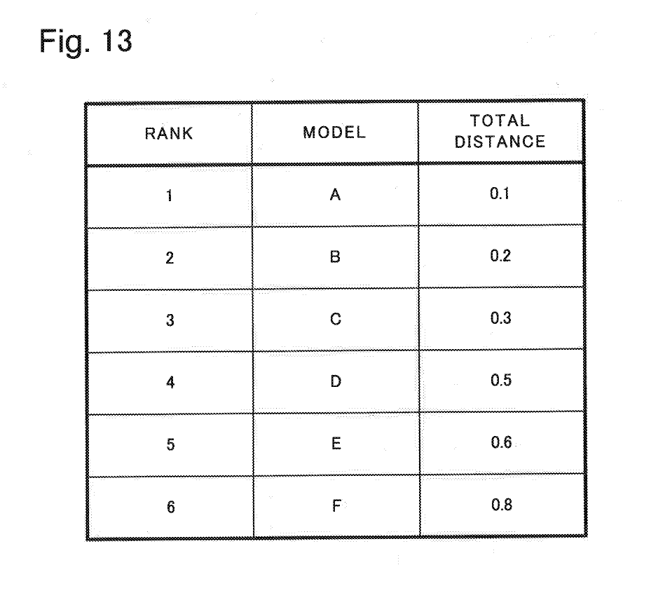

FIG. 13 is a diagram schematically illustrating an example of a stored degree of similarity. In the example illustrated in FIG. 13, a degree of similarity is a total distance. Then, the total distances are sorted in ascending order and are assigned with ranks.

The operation in Step S109 illustrated in FIG. 11 will be described in more detail by use of an example illustrated in FIG. 13. As described above, for example, the soil quality determination unit 105 may select as a monitoring model a soil quality model representing a quality of a soil of a model expressing the highest degree of similarity. In that case, in the example illustrated in FIG. 13, the soil quality determination unit 105 selects a soil quality model of a model A with the least total distance as a monitoring model.

When a measurement target soil is a soil in which a plurality of types of soil coexist, any one model may not necessarily be able to express the measurement target soil. As described above, the soil quality determination unit 105 may select a plurality of models in descending order of similarity indicated by a degree of similarity and generate a monitoring model, based on the selected models. Specifically, the soil quality determination unit 105 may assign a weight for each selected model, based on a degree of similarity of the selected model. The soil quality determination unit 105 may determine the weights in such a way that a sum of the assigned weights is equal to one. The soil quality determination unit 105 may multiply a parameter of a function expression expressing a soil quality model by an assigned weight, for each selected model. The soil quality determination unit 105 may generate a parameter of the function expression expressing the monitoring model by adding up parameters multiplied by weights for each parameter type (i.e. may generate a monitoring model).

For example, when generating a monitoring model by use of three models with high similarity in the example illustrated in FIG. 13, the soil quality determination unit 105 selects three models (models A, B, and C) in ascending order of total distance. Then, for example, the soil quality determination unit 105 may assign a weight proportional to a reciprocal of an obtained total distance to each of the selected models. For example, the soil quality determination unit 105 assigns a value obtained by dividing a reciprocal of a total distance of a class A by a sum of reciprocals of the respective total distances of classes A, B, and C as a weight of the class A. Similarly, for example, the soil quality determination unit 105 assigns a value obtained by dividing a reciprocal of the total distance of the class B by the sum of reciprocals of the respective total distances of the classes A, B, and C as a weight of the class B. For example, the soil quality determination unit 105 assigns a value obtained by dividing a reciprocal of the total distance of the class C by the sum of reciprocals of the respective total distances of the classes A, B, and C as a weight of the class C. In the example illustrated in FIG. 13, the total distance of the class A is 0.1, the total distance of the class B is 0.2, and the total distance of the class C is 0.3. Accordingly, the weight assigned to the model A is obtained as 6/11[=(1/0.1)/(1/0.1+1/0.2+1/0.3)]. The weight assigned to the model B is obtained as 3/11[=(1/0.2)/(1/0.1+1/0.2+1/0.3)]. The weight assigned to the model C is obtained as 2/11[=(1/0.3)/(1/0.1+1/0.2+1/0.3)].

Based on magnitude of a degree of similarity (a total distance in the example illustrated in FIG. 13), the soil quality determination unit 105 may determine whether to select a model with the highest similarity as a monitoring model or generate a monitoring model, based on a plurality of similar models. For example, when a degree of similarity indicating the highest similarity indicates a higher extent of similarity than a criterion (first criterion) indicated by a threshold value (first threshold value), the soil quality determination unit 105 may select the model with the highest similarity as a monitoring model. For example, when a degree of similarity indicating the highest similarity indicates an extent of similarity not higher than the first criterion indicated by the first threshold value, the soil quality determination unit 105 may generate a monitoring model, based on a plurality of models with high similarity indicated by a degree of similarity, as described above.

When a monitoring model is generated based on a plurality of models with high similarity indicated by a degree of similarity, the number of models used for the monitoring model may bepredetermined. When a monitoring model is generated based on a plurality of models with high similarity indicated by a degree of similarity, the soil quality determination unit 105 may select a plurality of models with similarity indicated by a degree of similarity being higher than a predetermined criterion (second criterion), by comparing the degree of similarity with a threshold value (second threshold value).

The present example embodiment described above provides the same effect as that provided by the first example embodiment. The reason is the same as the reason the effect according to the first example embodiment is provided.

### Fifth Example Embodiment

Next, a fifth example embodiment of the present invention will be described in detail with reference to a drawing.

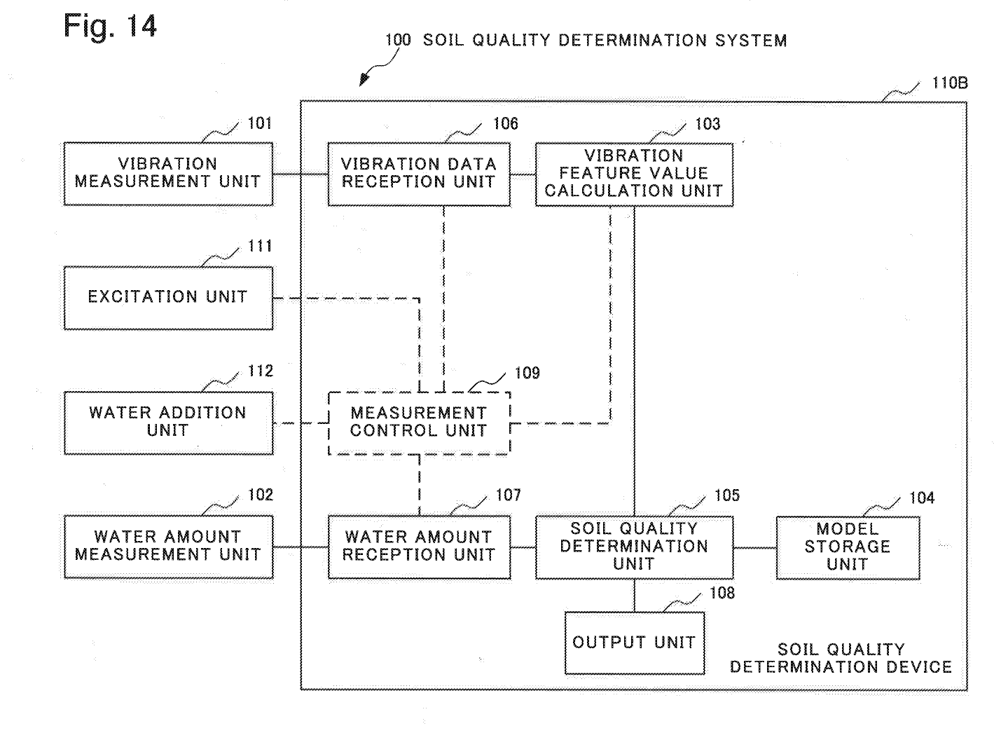

FIG. 14 is a block diagram illustrating a configuration example of a soil quality determination device 110B according to the present example embodiment.

Referring to FIG. 14, the soil quality determination device 110B according to the present example embodiment includes a vibration feature value calculation unit 103 and a soil quality determination unit 105. The soil quality determination device 110B calculates a vibration feature value, based on vibration data representing vibration of a target soil to which vibration is applied with repeated water addition. The soil quality determination unit 105 determines a quality of the target soil, based on a water amount-feature value distribution of the target soil, an extent of similarity of the water amount-feature value distribution between a soil type being a type of a soil from which the water amount-feature value distribution is obtained and the target soil, and a quality of the soil type. The water amount-feature value distribution represents a relation between a water amount measured when the vibration data are acquired and the vibration feature value.

The present example embodiment described above provides the same effect as that provided by the first example embodiment. The reason is the same as the reason the effect according to the first example embodiment is provided.

### Other Example Embodiments

For example, each of the soil quality determination device 110, 110A, and 110B, and the detection device 319, according to the aforementioned example embodiments, may be provided by a circuit. The circuit may be implemented as a single circuit. The circuit may be implemented as a plurality of circuits. The circuit may be implemented to be included in a single device. The circuit may be implemented by a plurality of devices.

For example, the circuit includes a processor and a memory. In that case, the processor executes a program loaded into the memory. The program is a program causing a computer including the processor and the memory to operate as the soil quality determination device 110, 110A, or 110B, or the detection device 319. Then, the computer including the processor and the memory operates as the soil quality determination device 110, 110A, and 110B.

For example, the circuit may be dedicated hardware. In that case, the dedicated hardware may include a circuit having a function of each component in the soil quality determination device 110, 110A, or 110B, or the detection device 319.

For example, the circuit may be a combination of the aforementioned computer and the aforementioned dedicated hardware.

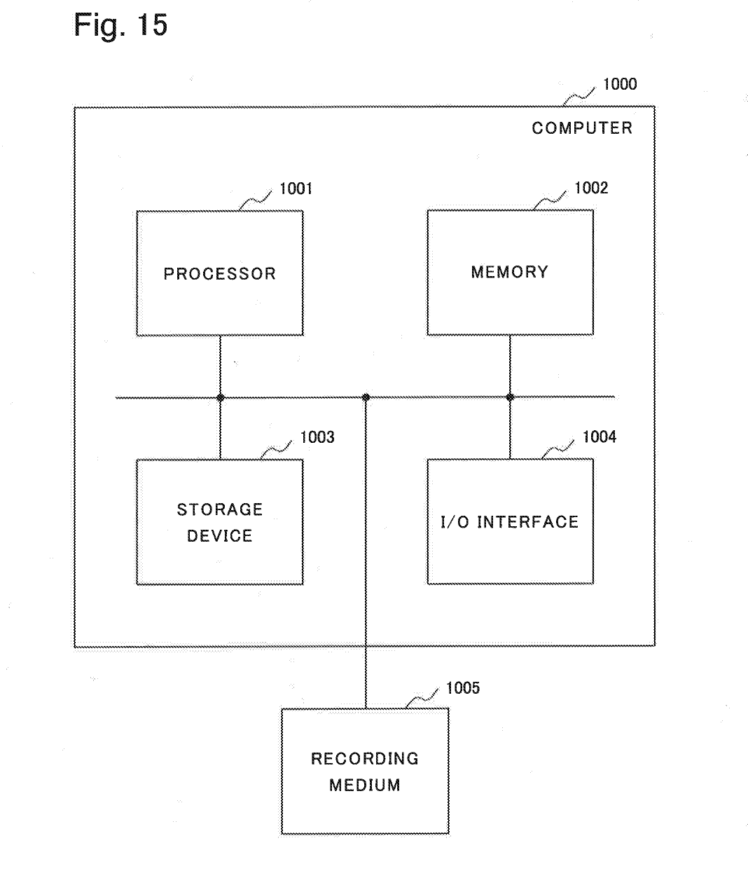

FIG. 15 is a diagram illustrating an example of a hardware configuration of a computer 1000 capable of providing the soil quality determination device and the detection device, according to the respective example embodiments of the present invention. Referring to FIG. 15, the computer 1000 includes a processor 1001, a memory 1002, a storage device 1003, and an input/output (I/O) interface 1004. Further, the computer 1000 is able to access a recording medium 1005. For example, the memory 1002 and the storage device 1003 include storage devices such as a random access memory (RAM) and a hard disk. For example, the recording medium 1005 includes storage devices such as a RAM and a hard disk, a read only memory (ROM), and a portable recording medium. The storage device 1003 may be the recording medium 1005. The processor 1001 is able to read and write data and a program from and to the memory 1002 and the storage device 1003. For example, the processor 1001 is able to access the vibration measurement unit 101 and the water amount measurement unit 102 through the I/O interface 1004. The processor 1001 is able to access the recording medium 1005. The recording medium 1005 stores a program causing the computer 1000 to operate as the soil quality determination device 110, 110A, or 110B.

The processor 1001 loads into the memory 1002 a program causing the computer 1000 to operate as the soil quality determination device 110, 110A, or 110B, the program being stored in the recording medium 1005. Then, by the processor 1001 executing the program loaded in the memory 1002, the computer 1000 operates as the soil quality determination device 110, 110A, or 110B.

For example, each component included in a first group below can be provided by the memory 1002 in which a dedicated program capable of providing a function of the component is loaded and the processor 1001 executing the program. The aforementioned first group includes the vibration feature value calculation unit 103, the soil quality determination unit 105, the vibration data reception unit 106, the water amount reception unit 107, the output unit 108, and the measurement control unit 109. The aforementioned first group further includes the adhesive strength-internal friction angle calculation module 306, the adhesive strength-internal friction angle modeling module 307, the water content associating module 308, the vibration feature value calculation module 309, the weight-pore water pressure modeling module 310, the soil quality determination module 314, and the slope safety factor calculation determination module 315.

Further, the model storage unit 104 and the database 311 can be provided by the memory 1002 and the storage device 1003 such as a hard disk device that are included in the computer 1000.

The respective components included in the aforementioned first group, the model storage unit 104, and the database 311 can be also provided by dedicated circuits providing the functions thereof.

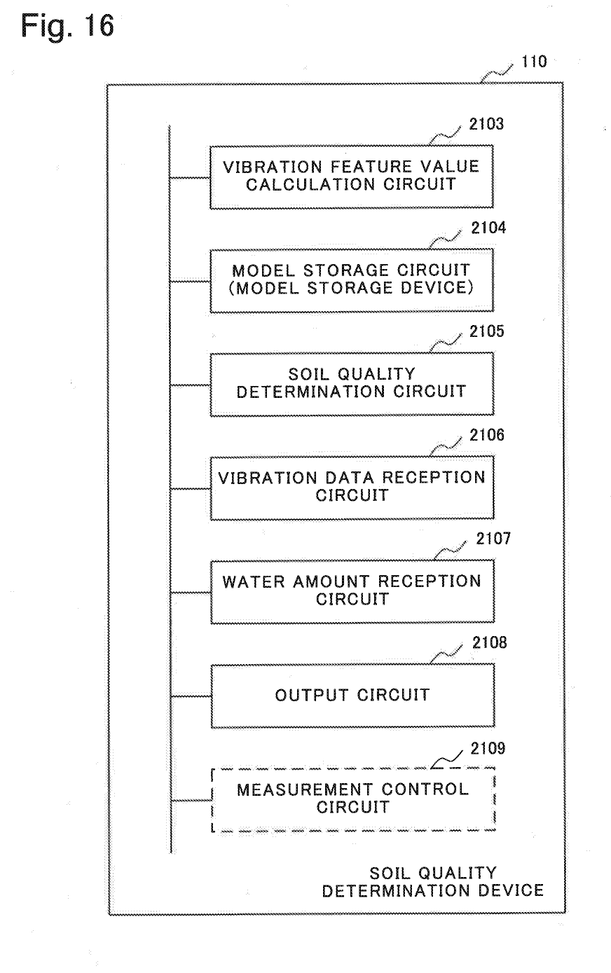

FIG. 16 is a block diagram illustrating a configuration example of the soil quality determination device 110 according to the first example embodiment, the device being implemented by use of dedicated circuits. The soil quality determination device 110A according to the second and fourth example embodiments may be implemented similarly to the soil quality determination device 110 illustrated in FIG. 16.

Referring to FIG. 16, the soil quality determination device 110 includes a vibration feature value calculation circuit 2103, a model storage circuit 2104, a soil quality determination circuit 2105, a vibration data reception circuit 2106, a water amount reception circuit 2107, and an output circuit 2108. The soil quality determination device 110 may further include a measurement control circuit 2109. The vibration feature value calculation circuit 2103 operates as the vibration feature value calculation unit 103. The model storage circuit 2104 operates as the model storage unit 104. For example, the model storage unit 104 may be implemented by a storage device such as a hard disk device or a solid state disk (SSD). The soil quality determination circuit 2105 operates as the soil quality determination unit 105. The vibration data reception circuit 2106 operates as the vibration data reception unit 106. The water amount reception circuit 2107 operates as the water amount reception unit 107. The output circuit 2108 operates as the output unit 108. The measurement control circuit 2109 operates as the measurement control unit 109.

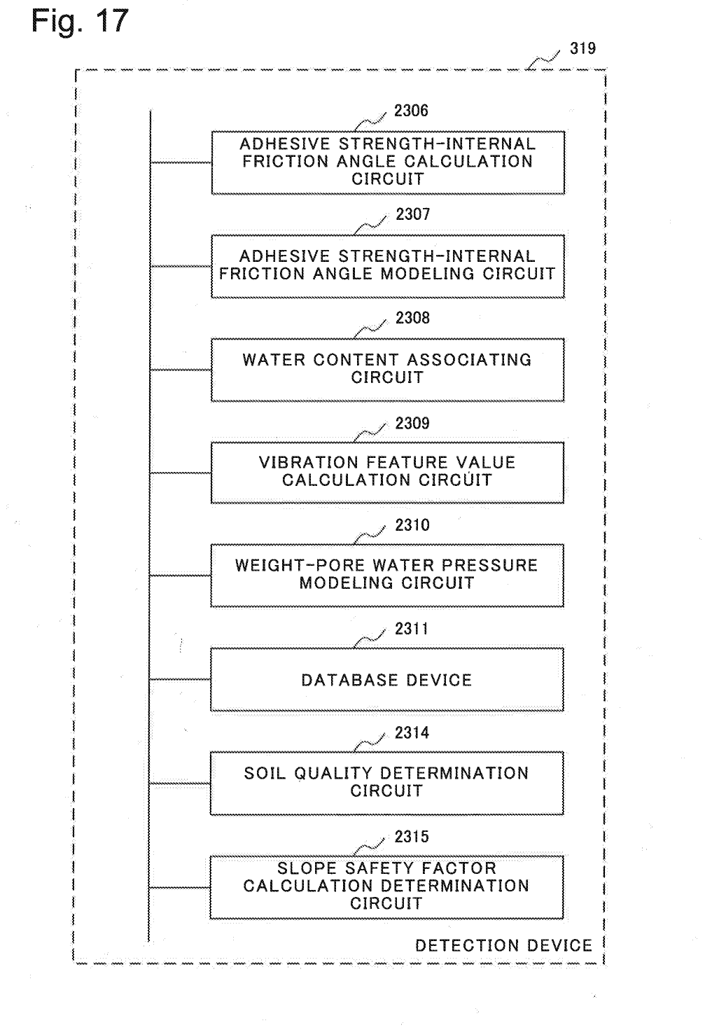

FIG. 17 is a block diagram illustrating a configuration example of the detection device 319 according to the third example embodiment, the device being implemented by use of dedicated circuits.

Referring to FIG. 17, the detection device 319 includes an adhesive strength-internal friction angle calculation circuit 2306, an adhesive strength-internal friction angle modeling circuit 2307, a water content associating circuit 2308, a vibration feature value calculation circuit 2309, and a weight-pore water pressure modeling circuit 2310. The detection device 319 further includes a database device 2311, a soil quality determination circuit 2314, and a slope safety factor calculation determination circuit 2315.

The adhesive strength-internal friction angle calculation circuit 2306 operates as the adhesive strength-internal friction angle calculation module 306. The adhesive strength-internal friction angle modeling circuit 2307 operates as the adhesive strength-internal friction angle modeling module 307. The water content associating circuit 2308 operates as the water content associating module 308. The vibration feature value calculation circuit 2309 operates as the vibration feature value calculation module 309. The weight-pore water pressure modeling circuit 2310 operates as the weight-pore water pressure modeling module 310. The database device 2311 operates as the database 311. The soil quality determination circuit 2314 operates as the soil quality determination module 314. The slope safety factor calculation determination circuit 2315 operates as the slope safety factor calculation determination module 315.

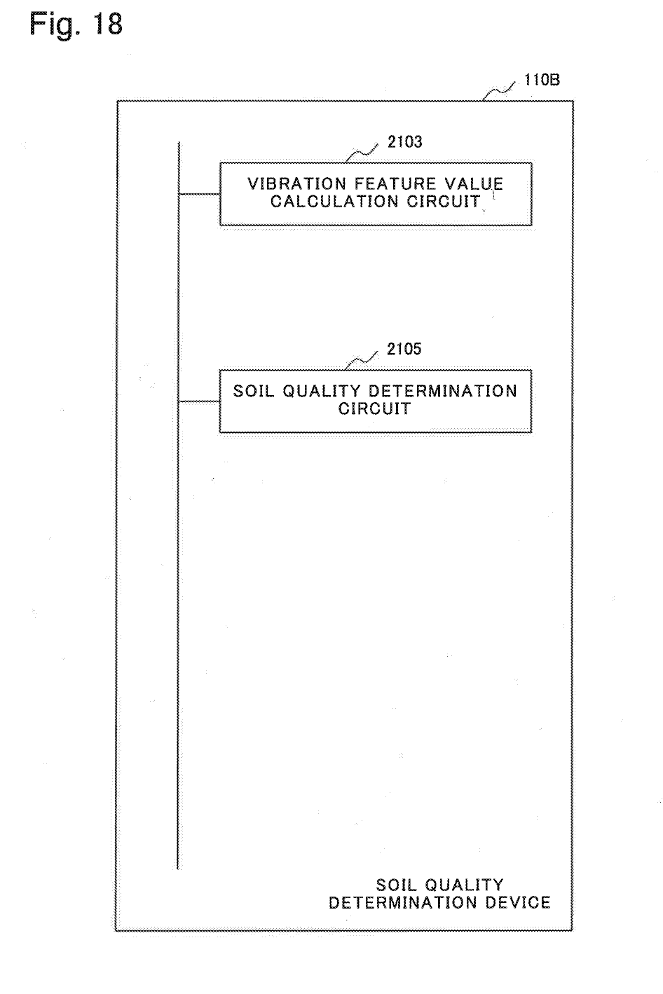

FIG. 18 is a block diagram illustrating a configuration example of the soil quality determination device 110B according to the fifth example embodiment, the device being implemented by use of dedicated circuits.

Referring to FIG. 18, the soil quality determination device 110B includes a vibration feature value calculation circuit 2103 and a soil quality determination circuit 2105. The vibration feature value calculation circuit 2103 operates as the vibration feature value calculation unit 103. The soil quality determination circuit 2105 operates as the soil quality determination unit 105.

While the present invention has been described above with reference to the example embodiments, the present invention is not limited to the aforementioned example embodiments. Various changes and modifications that can be understood by a person skilled in the art may be made to the configurations and details of the present invention, within the scope of the present invention.

This application claims priority based on Japanese Patent Application No. 2015-193107 filed on Sep. 30, 2015, the disclosure of which is hereby incorporated by reference thereto in its entirety.

### REFERENCE SIGNS LIST

100 Soil quality determination system

100A Soil quality determination system

101 Vibration measurement unit

102 Water amount measurement unit

103 Vibration feature value calculation unit

104 Model storage unit

105 Soil quality determination unit

106 Vibration data reception unit

107 Water amount reception unit

108 Output unit

109 Measurement control unit

110 Soil quality determination device

110A Soil quality determination device

110B Soil quality determination device

111 Excitation unit

112 Water addition unit

301 Stress sensor

302 Stress sensor

303 Moisture meter

304 Vibration sensor

305 Pore water pressure meter

306 Adhesive strength-internal friction angle calculation module

307 Adhesive strength-internal friction angle modeling module

308 Water content associating module

309 Vibration feature value calculation module

310 Weight-pore water pressure modeling module

311 Database

312 Vibration sensor

313 Moisture meter

314 Soil quality determination module

315 Slope safety factor calculation determination module

316 Display

317 Triaxial compression testing device

318 Planter

319 Detection device

320 Actual slope measurement device

1000 Computer

1001 Processor

1002 Memory

1003 Storage device

1004 I/O interface

1005 Recording medium

2103 Vibration feature value calculation circuit

2104 Model storage circuit

2105 Soil quality determination circuit

2106 Vibration data reception circuit

2107 Water amount reception circuit

2108 Output circuit

2109 Measurement control circuit

2306 Adhesive strength-internal friction angle calculation circuit

2307 Adhesive strength-internal friction angle modeling circuit

2308 Water content associating circuit

2309 Vibration feature value calculation circuit

2310 Weight-pore water pressure modeling circuit

2311 Database device

2314 Soil quality determination circuit

2315 Slope safety factor calculation determination circuit

What is claimed is:

1. A soil quality determination device comprising:
a memory that stores a set of instructions; and
at least one processor configured to execute the set of instructions to:
calculate a vibration feature value, based on vibration data representing vibration of a target soil to which vibration is applied with repeated water addition; and
determine a quality of the target soil, based on a water amount-feature value distribution of the target soil, the distribution representing a relation between a water amount measured when the vibration data are acquired and the vibration feature value, an extent of similarity of the water amount-feature value distribution between a soil type being a type of a soil from which the water amount-feature value distribution is obtained and the target soil, and a quality of the soil type.

2. The soil quality determination device according to claim 1, wherein
the at least one processor is configured to:
calculate, based on the vibration data on which frequency filtering of passing a signal at a frequency included in a pass frequency band is performed for each of a plurality of pass frequency bands, the vibration feature value for each of the pass frequency bands; and
determine the quality of the target soil, based on an extent of similarity of the water amount-feature value distribution for each of the pass frequency bands between the soil type from which the water amount-feature value distribution for each of the pass frequency bands is obtained and the target soil, and a quality of the soil type.

3. The soil quality determination device according to claim 1, wherein
the at least one processor is configured to:
select, based on extents of similarity between the water amount-feature value distribution of the target soil and the water amount-feature value distributions of a plurality of soil types, one soil type from the plurality of soil types;
determine a mixing ratio of a soil of the selected soil type, based on the extents of similarity;
estimate a quality of a soil into which a soil of the selected soil type is mixed, the soil of the selected soil type being mixed at a determined mixing ratio; and
determine an estimated quality to be a quality of the target soil.

4. The soil quality determination device according to claim 1, wherein
the vibration feature value is a damping factor, and
the at least one processor is configured to
determine a quality of the target soil, the quality including a density, based on the water amount-feature value distribution of the soil type for a plurality of combinations of the soil type and a density.

5. A soil quality determination system comprising:
the soil quality determination device according to claim 1;
a vibration measurement device that measures vibration of the target soil and outputting the vibration data representing measured vibration; and
a water amount measurement device that measures a water amount of the target soil.

6. A soil quality determination method comprising:
calculating a vibration feature value, based on vibration data representing vibration of a target soil to which vibration is applied with repeated water addition; and
determining a quality of the target soil, based on a water amount-feature value distribution of the target soil, the distribution representing a relation between a water amount measured when the vibration data are acquired and the vibration feature value, an extent of similarity of the water amount-feature value distribution between a soil type being a type of a soil from which the water amount-feature value distribution is obtained and the target soil, and a quality of the soil type.

7. The soil quality determination method according to claim 6, further comprising:
based on the vibration data on which band-pass filtering is performed for each of a plurality of pass frequency bands, calculating the vibration feature value for each of the pass frequency bands; and
determining the quality of the target soil, based on an extent of similarity of the water amount-feature value distribution for each of the pass frequency bands between the soil type from which the water amount-feature value distribution for each of the pass frequency bands is obtained and the target soil, and a quality of the soil type.

8. The soil quality determination method according to claim 6, further comprising:
based on extents of similarity between the water amount-feature value distribution of the target soil and the water amount-feature value distributions of a plurality of soil types, selecting one soil type from the plurality of soil types;
determining a mixing ratio of a soil of a selected soil type, based on the extents of similarity;
estimating a quality of a soil into which a soil of the selected soil type is mixed, the soil of the selected soil type being mixed at a determined mixing ratio; and
determining an estimated quality to be a quality of the target soil.

9. A non-transitory computer readable storage medium storing a soil quality determination program causing a computer to execute:
vibration feature value calculation processing of calculating a vibration feature value, based on vibration data representing vibration of a target soil to which vibration is applied with repeated water addition; and
soil quality determination processing of determining a quality of the target soil, based on a water amount-feature value distribution of the target soil, the distribution representing a relation between a water amount measured when the vibration data are acquired and the vibration feature value, an extent of similarity of the water amount-feature value distribution between a soil type being a type of a soil from which the water amount-feature value distribution is obtained and the target soil, and a quality of the soil type.

10. The storage medium according to claim 9, storing the soil quality determination program, wherein,
based on the vibration data on which band-pass filtering is performed for each of a plurality of pass frequency bands, the vibration feature value calculation processing calculates the vibration feature value for each of the pass frequency bands, and
the soil quality determination processing determines the quality of the target soil, based on an extent of similarity of the water amount-feature value distribution for each of the pass frequency bands between the soil type from which the water amount-feature value distribution for each of the pass frequency bands is obtained and the target soil, and a quality of the soil type.
